SEQUENCE LISTING (7 sequences)

SEQ ID NO: 1 - protein, 84 aa, Homo sapiens
  PEPTIDE (1)...(84): parathyroid hormone peptide
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 84 aa, Rat rattus
  PEPTIDE (1)...(84): parathyroid hormone peptide
AVSEIQLMHNLGKHLASVERMQWLRKKLQDVHNFVSLGVQMAAREGSYQRPTKKEDNVLVDGNSKSLGEGDKADVDVLVKAKSQ

SEQ ID NO: 3 - protein, 84 aa, Mus musculus
  PEPTIDE (1)...(84): parathyroid hormone peptide
AVSEIQLMHNLGKHLASVERMQWLRRKLQDMHNFVSLGVQMAARDGSHQKPTKKEENVLVDGNPKSLGEGDKADVDVLVKSKSQ

SEQ ID NO: 4 - protein, 84 aa, Bos taurus
  PEPTIDE (1)...(84): parathyroid hormone peptide
AVSEIQFMHNLGKHLSSMERVEWLRKKLQDVHNFVALGASIAYRDGSSQRPRKKEDNVLVESHQKSLGEADKADVDVLIKAKPQ

SEQ ID NO: 5 - protein, 84 aa, Canis lupus
  PEPTIDE (1)...(84): parathyroid hormone peptide
SVSEIQFMHNLGKHLSSMERVEWLRKKLQDVHNFVALGAPIAHRDGSSQRPLKKEDNVLVESYQKSLGEADKADVDVLTKAKSQ

SEQ ID NO: 6 - protein, 84 aa, Sus domestica
  PEPTIDE (1)...(84): parathyroid hormone peptide
SVSEIQFMHNLGKHLSSLERVEWLRKKLQDVHNFVALGASIVHRDGGSQRPRKKEDNVLVESHQKSLGEADKAAVDVLIKAKPQ

SEQ ID NO: 7 - protein, 86 aa, Equus caballus
  PEPTIDE (1)...(86): parathyroid hormone peptide
KRSVSEIQLMHNLGKHLNSVERVEWLRKKLQDVHNFIALGAPIFHRDGGSQRPRKKEDNVLIESHQXSLGEADKADVDVLSKTKSQ

(19) United States
(12) Patent Application Publication
(10) Pub. No.: US 2010/0021496 A1
(43) Pub. Date: Jan. 28, 2010

(54) METHODS, KITS, AND ANTIBODIES FOR QUANTITATIVE DETERMINATION OF PARATHYROID HORMONE MOLECULES WITH INTACT C-TERMINUS

(76) Inventors: Thomas L. CANTOR, El Cajon, CA (US); Zan W. Yang, Santee, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/423,702

(22) Filed: Apr. 14, 2009

Related U.S. Application Data
(60) Provisional application No. 61/045,267, filed on Apr. 15, 2008.

(51) Int. Cl. A61K 39/00; C07K 16/26; G01N 33/53; C07K 14/575

(52) U.S. Cl. 424/198.1; 530/387.1; 530/388.24; 530/389.2; 435/7.1; 435/7.94; 435/7.93; 435/7.5; 530/399

(57) ABSTRACT

The present invention relates to novel methods and compositions useful for detecting whole parathyroid hormone at a physiological level and C-terminal parathyroid fragments in a mammalian sample. Such detections may be useful to different parathyroid diseases or disorders in a subject, such as chronic renal failure, hyperparathyroidism and related bone diseases, from normal or non-disease states. One detects whole or non-fragmented (1 to 84) parathyroid hormone in a biological sample and optionally one or more of a selection of C-terminal parathyroid hormone peptide fragments that may or may not function as a parathyroid hormone antagonists. By either comparing values or using independently the value of either the one or more of a selection of C-terminal parathyroid hormone peptide fragments, the whole parathyroid hormone, or the combination of these values, one is able to differentiate chronic renal failure, parathyroid and bone related disease states, as well as differentiate such states from normal states.

### I. CROSS-REFERENCE TO RELATED APPLICATIONS

This application claims priority from U.S. provisional application 61/045,267 filed Apr. 15, 2008. The contents of this document are incorporated herein by this reference.

### II. TECHNICAL FIELD

The present invention relates to novel compositions, methods and kits for differentiating parathyroid diseases in a subject. These compositions, methods and kits can be used, for example to differentiate hyperparathyroidism, chronic renal failure, high bone turnover, and adynamic bone disease from normal or non-disease states.

### III. BACKGROUND OF THE INVENTION

Calcium plays an indispensable role in cell permeability, the formation of bones and teeth, blood coagulation, transmission of nerve impulse, and normal muscle contraction. The concentration of calcium ions in the blood is, along with calcitrol and calcitonin, regulated mainly by parathyroid hormone (PTH). Although calcium intake and excretion may vary, PTH serves through a feedback mechanism to maintain a steady concentration of calcium in cells and surrounding fluids. When serum calcium lowers, the parathyroid glands secrete PTH, affecting the release of stored calcium. When serum calcium increases, stored calcium release is retarded through lowered secretions of PTH.

The complete form of human PTH, (hPTH), is a unique 84 amino acid peptide (SEQ ID NO: 1), as is shown in FIG. 1. Researchers have found that this peptide has an anabolic effect on bone that involves a domain for protein kinase C activation (amino acid residues 28 to 34) as well as a domain for adenylate cyclase activation (amino acid residues 1 to 7). However, various catabolic forms of clipped or fragmented PTH peptides also are found in circulation, most likely formed by intraglandular or peripheral metabolism. For example, whole PTH can be cleaved between amino acids 34 and 35 to produce a (1-34) PTH N-terminal fragment and a (35-84) PTH C-terminal fragment. A large PTH fragment referred to as “non-(1-84) PTH” has also been disclosed which is clipped closer to the N-terminal end of PTH ((7-84) PTH). (See LePage, R., et al., Clin. Chem. 44: 805-810 (1998)). Recently, (34-84) PTH, (37-84) PTH, (38-84) PTH and (45-84) PTH C-terminal fragments have been identified from human plasma. (See Zhang C. X., et al., Anal. Chem. 78(5): 1639-1643 (2005)).

The total human C-terminal PTH fragments constitute approximately 80% of circulating PTH. (See D'Amour P. & Brossard J-H., Nephrol. Hyperten. 14: 330-336 (2005)). Synthetic C-terminal PTH fragments were reported to be able to activate the C-terminal PTH receptors and decrease the high bone turnover. Administration of C-terminal PTH fragments, including (7-84) PTH, in rats has made possible the demonstration that C-terminal PTH has inverse biological actions to PTH (1-84). (See Divieti P., et al., Endocrinology 143(1): 171-176 (2002); Langub M. C., et al., Endocrinology 144(4): 1135-1138 (2003)). Accumulation of C-terminal PTH fragments in renal failure patients may cause PTH resistance and may be associated with adynamic bone disease (See D'Amour P. & Brossard J-H., Nephrol. Hyperten. 14: 330-336 (2005)). Past studies demonstrated that C-terminal PTH stimulated osteoclast-like cell formation and osteoclastic activity (See Kaji H., et al., J. Bone Min. Metab. 12: S125-S129 (2006)). The C-terminal PTH fragments also were reported to be associated with alkaline phosphatase activity in rat osteoblastic cells ROS 17/2.8. Those fragments were demonstrated to inhibit the binding of PTH (1-84) to the ROS 17/2.8 cells. A short C-terminal fragment, PTH (71-84), still retained its inhibitory effect (See Takasu H., et al., J. Bone and Min., 12: S131-S134 (1994)).

The clinical need for accurate measurement of PTH is well demonstrated. Serum PTH level is one of the most important index for patients with the following diseases: familial hypocalciuric hypercalcemia; multiple endocrine neoplasia types I and II; osteoporosis; Paget's bone disease; primary hyperparathyroidism—caused by primary hyperplasia or adenoma of the parathyroid glands; pseudohypoparathyroidism; and renal failure, which can cause secondary hyperparathyroidism.

PTH plays a role in the course of disease in a patient with chronic renal failure. Renal osteodystrophy (RO) is a complex skeletal disease comprising osteitis fibrosa cystica (caused by PTH excess), osteomalacia-unmineralized bone matrix (caused by vitamin D deficiency), extraskeletal calcification/ossification (caused by abnormal calcium and phosphorus metabolism), and adynamic bone disease (contributed to by PTH suppression). Chronic renal failure patients can develop RO. Failing kidneys increase serum phosphorus (hyperphosphoremia) and decrease 1,25-dihydroxyvitamin D (1,25-D) production by the kidney. The former results in secondary hyperparathyroidism from decreased gastrointestinal calcium absorption and osteitis fibrosa cystica from increased PTH in response to an increase in serum phosphorus. The later causes hypocalcemia and osteomalacia. With the onset of secondary hyperparathyroidism, the parathyroid gland becomes less responsive to its hormonal regulators because of decreased expression of its calcium and vitamin D receptors. Serum calcium drops. RO can lead to digital gangrene, bone pain, bone fractures, and muscle weakness.

An important discovery leading to the present invention is that adynamic bone loses its capacity to buffer calcium and phosphate as the bones are shut down. In subjects afflicted with such conditions, they are unable to effectively buffer calcium as it enters their bodies through their diet. This calcium enters the blood stream and is thereafter shuttled to the soft tissues. The parathyroid gland is particularly subject to, and detrimentally affected by, this influx of calcium and thereby produces PTH fragments rather than, or in addition to, the active form of PTH. Accordingly, in subjects with adynamic bone, the concentration and production of PTH fragments is increased.

Determining circulating biologically active PTH levels in humans has been challenging. One major problem is that PTH is found at low levels, normally 10 pg/mL to 40 pg/mL (i.e., 1 pmol/L to 4 pmol/L). Coupled with extremely low circulating levels is the problem of the heterogeneity of PTH and its many circulating fragments. In many cases, immunoassays have faced substantial and significant interference from circulating PTH fragments. For example, some commercially available PTH kits have almost 100% cross-reactivity with the non-(1-84) PTH fragment. See the LePage article supra.

PTH immunoassays have varied over the years. One early approach is a double antibody precipitation immunoassay found in U.S. Pat. No. 4,369,138, issued to Arnold W. Lindall et alia. A first antibody has a high affinity for a (65-84) PTH fragment. A radioactive labeled (65-84) PTH peptide is added to the sample with the first antibody to compete for the unlabeled peptide. A second antibody is added which binds to any first antibody and radioactive labeled PTH fragment complex, thereby forming a precipitate. Both precipitate and supernatant can be measured for radioactive activity, and PTH levels can be calculated therefrom.

In an effort to overcome PTH fragment interference, immunoradiometric two-site assays for intact PTH (I-PTH) have been introduced, such as Allegro® Intact PTH assay by the Nichols Institute of San Juan Capistrano, Calif. In one version, a capture antibody specifically binds to the C-terminal portion of hPTH while a labeled antibody specifically binds to the N-terminal portion of the captured hPTH. In another, two monoclonal antibodies were used, both of which attached to the N-terminal portion of hPTH. (For the purposes of the present disclosure, the complete form of human PTH is referred to as “whole PTH” or “wPTH” as distinguished from “intact PTH” or “I-PTH” which can include not only wPTH, but also a large PTH fragment cleaved about amino acids 5 to 8.) Unfortunately, these assays have problems in that they measure but do not discriminate between w-PTH and I-PTH. This inability comes to the fore in hyperparathyroid patients and renal failure patients who have significant endogenous concentrations of large, non-whole PTH fragments.

To overcome some of these difficulties, Scantibodies Laboratories, Inc. (Santee, Calif., U.S.A.) previously developed a method of quantifying whole PTH, which utilizes an antibody to (1-6) PTH to detect only PTH peptides having an intact N-terminus and thus avoids detecting the non-whole (7-84) PTH fragment and other large C-terminal fragments (see U.S. Patent Application No. 2004/0219598 A1, published Nov. 4, 2004, the content of which is incorporated herein). However, one of the shortcomings of the whole PTH assay is that, much like all the other PTH assays, it is unable to differentiate between PTH variants with an intact C-terminus and those in which one or more C-terminal amino acids are missing.

As discussed earlier, C-terminal PTH fragments account for approximately 80% of circulating PTH and have been shown to decrease high bone turnover by activating the C-terminal PTH receptors. Moreover, it has been demonstrated that C-terminal PTH fragments antagonize the effect of whole PTH in rats and stimulate osteoclast-like cell formation and osteoclastic activity. The C-terminal PTH fragments have also been reported to be associated with alkaline phosphatase activity in rat osteoblasts. It has been hypothesized that accumulation of C-terminal PTH fragments in human renal failure patients may cause PTH resistance and may be associated with adynamic bone disease. In light of this and other related information, the measurement of C-terminal PTH fragment levels, and particularly in conjunction with the measurement of whole PTH, can be used effectively to differentiate subjects having adynamic bone versus those having normal bone and high bone turnover rates, and to provide better diagnosis and treatment of subjects experiencing chronic renal failure.

There is a compelling need to be able to non-invasively separate the dialysis patients with ADN from those suffering from high bone turnover to avoid over treatment of ADN dialysis patients. Over treatment of dialysis patients with ADN is a frequent occurrence under presently utilized methods. For example, package inserts that proscribe the use of Zemplar® and Calcijex® (Abbott Laboratories), for example, are being used to treat thousands of dialysis patients that stand a great risk of over treatment under the proscribed protocols that do not account for circulating total PTH fragment levels. The present invention addresses these and other needs in the art.

### IV. DISCLOSURE OF THE INVENTION

In one embodiment, the present disclosure provides an isolated antibody that specifically binds to a C-terminal sequence of whole parathyroid hormone (PTH). Preferably, the isolated antibody is capable of detecting said whole PTH, a non-whole PTH fragment and a C-terminal PTH fragment at a physiological level in a mammalian sample, and/or specifically binds to a whole PTH, a non-whole PTH fragment and a C-terminal PTH fragment having an intact C-terminus. Frequently, the isolated antibody is a monoclonal or polyclonal antibody. Also frequently, the binding between the antibody and the C-terminal sequence of the whole PTH, non-whole PTH fragment(s) and C-terminal PTH fragment(s) is dependent on the presence of amino acid residues 75-84 or 75-86 of the PTH.

In one aspect an isolated antibody of the present disclosure specifically binds to an epitope comprised in PTH₈₁₋₈₄, PTH₈₀₋₈₄, PTH₇₉₋₈₄, PTH₇₈₋₈₄, PTH₇₇₋₈₄, PTH₇₆₋₈₄, PTH₇₅₋₈₄, PTH₇₄₋₈₄, PTH₇₃₋₈₄, PTH₇₂₋₈₄, PTH₇₁₋₈₄ or PTH₇₀₋₈₄. Frequently, an isolated antibody of the present disclosure specifically binds to the parathyroid hormone peptide PTH₇₅₋₈₄.

In a further embodiment a multiple antigenic peptide (MAP) is provided, which MAP comprises a branched oligolysine core conjugated with a plurality of a PTH peptide as described herein. On occasion, the branched oligolysine core comprises 3, 7 or 15 lysine residues, also on occasion, the MAP comprises 4, 8 or 16 copies of the PTH peptide. The plurality of the PTH peptide comprises the same or different PTH peptides. In one aspect, the plurality of the PTH peptide is conjugated to the branched oligolysine core via a spacer. Frequently, the spacer is an amino acid residue. Multiple antigenic peptides comprise generally known technology. See, e.g., Adermann, K., et al., Innovations and Perspectives in Solid Phase Synthesis, 429-32 (R. Epton, ed., Mayflower Worldwide 1994).

In another embodiment, the present disclosure provides a method for measuring the level of a whole PTH, a non-whole PTH fragment and/or a C-terminal PTH fragment having an intact C-terminus in a mammalian sample, which method comprises: a) obtaining a sample from a mammal to be tested; b) contacting said sample with an isolated antibody that specifically binds to a C-terminal sequence of whole PTH, preferably, the isolated antibody being capable of detecting said whole PTH, non-whole PTH fragment and/or C-terminal PTH fragment at a physiological level in said mammalian sample, and/or specifically binding to said whole PTH, non-whole PTH fragment and/or C-terminal PTH fragment having an intact C-terminus; and c) assessing a complex formed between said whole parathyroid hormone, non-whole PTH fragment and/or C-terminal PTH fragment, if present in said sample, and said antibody, to measure the level of said whole PTH, non-whole PTH fragment and/or C-terminal PTH fragment in said mammalian sample.

In one aspect an isolated antibody of the present disclosure specifically binds to an epitope comprised in PTH₈₁₋₈₄, PTH₈₀₋₈₄, PTH₇₉₋₈₄, PTH₇₈₋₈₄, PTH₇₇₋₈₄, PTH₇₆₋₈₄, PTH₇₅₋₈₄, PTH₇₄₋₈₄, PTH₇₃₋₈₄, PTH₇₂₋₈₄, PTH₇₁₋₈₄ or PTH₇₀₋₈₄. Frequently, an isolated antibody of the present disclosure specifically binds to the parathyroid hormone peptide PTH₇₅₋₈₄.

Although a variety of assay types are contemplated, the present methods frequently assess the complex formed between the whole parathyroid hormone or C-terminal PTH fragments and the antibody via a sandwich or competitive assay format. On occasion, the complex is assessed in a homogeneous or a heterogeneous assay format. Also frequently, the complex is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, u-capture assay, inhibition assay and avidity assay. In a sandwich assay format, the antibody that specifically binds to a C-terminal sequence of whole PTH is used as a first antibody and antibody that is capable of binding to a portion of whole PTH other than the C-terminal sequence which binds to the first antibody is used as a second antibody. Either the first antibody or the second antibody is frequently attached to a surface and functions as a capture antibody. The attachment can be direct or indirect. In a preferred embodiment, the attachment is provided via a biotin-avidin (or streptavidin) linking pair.

In another aspect, the physiological level of whole parathyroid hormone is less than 4 pmol/L. Frequently, the physiological level of whole parathyroid hormone is from about 0.2 pmol/L to about 4 pmol/L. Also frequently, the physiological range of whole PTH ranges between about 2 pg/ml to about 40 pg/ml. On occasion, the physiological range of whole PTH ranges between about 7 pg/ml to about 39 pg/ml.

In a further embodiment, the present methods may be utilized to measure multiple PTH peptide components, such as a whole PTH level (wPTH) and/or a total PTH level (tPTH), in addition to a C-terminal PTH level (cPTH). In such embodiments, the methods frequently further comprise comparing at least two parameters selected from the group consisting of the whole PTH level, total PTH level and C-terminal PTH level. The comparison of parameters is generally in the form of a ratio or proportion. Frequently, the results of said comparison are used to determine whether the mammal, often comprising a human patient, is afflicted with a bone turnover related disorder, or used to monitor bone disease related treatment. Also frequently, the present methods are used to determine or diagnose whether the mammal is afflicted with, or at risk for, adynamic bone disease or severe hyperparathyroidism. Frequently, the present methods are used for clinical management of renal disease subjects and subjects afflicted with osteoporosis, including dialysis patients. Also frequently, the present methods are used for diagnosing primary hyperparathyroidism. Moreover, the present methods are useful for clinical diagnosis and management of subjects having adynamic bone disease induced, in part, through the practice of inappropriate treatment protocols.

In preferred embodiments of the present comparison the comparison takes many forms. For example, the comparison can be in the form of a ratio or proportion between the C-terminal PTH level versus the whole PTH level (i.e., represented by the equation: cPTH/wPTH); between the C-terminal PTH level versus the total PTH levels (i.e., represented by the equation: cPTH/total PTH); or other combinations of the disclosed parameters, including, without limitation, the inverse of each comparison. The cutoff ranges for each of these comparisons may vary as they are associated with a particular bone turnover, treatment, disease or disorder.

Frequently in the present methods the sample is contacted with one or more isolated antibodies, and wherein each of said one or more isolated antibodies specifically binds one or more PTH peptide fragments selected from the group consisting of: PTH₄₀₋₆₀, PTH₃₅₋₄₀, PTH₅₃₋₇₃, and PTH₃₅₋₇₄.

The present methods of measuring multiple PTH components provide a variety of uses. For example, such methods are used for differentiating between a person having substantially normal parathyroid function and having hyperparathyroidism, e.g., primary hyperparathyroidism; monitoring parathyroid related bone disease and treatment; monitoring effects of therapeutic treatment for hyperparathyroidism; diagnosing parathyroid related bone disease; and diagnosing, monitoring and treatment of renal disease subjects and subjects afflicted with osteoporosis.

The present disclosure further provides kits for measuring the level of a whole PTH, a non-whole PTH fragment and/or a C-terminal PTH fragment having an intact C-terminus in a mammalian sample, which kit comprises, in a container, an isolated antibody that specifically binds to a C-terminal sequence of whole parathyroid hormone (PTH); preferably, the isolated antibody is capable of detecting said whole PTH, a non-whole PTH fragment and/or a C-terminal PTH fragment at a physiological level in a mammalian sample, and/or specifically binds to a whole PTH, a non-whole PTH fragment and a C-terminal PTH fragment having an intact C-terminus. In one embodiment, a kit for measuring a physiological level of whole parathyroid hormone (PTH), non-whole PTH fragment(s) and/or C-terminal PTH fragment(s) in a mammalian sample.

In another embodiment, the present disclosure further provides kits for producing an antibody to a parathyroid hormone (PTH) or a PTH peptide, which kits comprise: a) an isolated PTH peptide; b) means for introducing said isolated PTH peptide to a mammal in an amount sufficient to produce an antibody to said PTH peptide; and c) means for recovering said antibody from said mammal. In further embodiment, a kit for producing an antibody to a parathyroid hormone (PTH) or a PTH peptide is provided which comprises: a) a MAP; b) a means for introducing said MAP to a mammal in an amount sufficient to produce an antibody to a PTH peptide comprised in said MAP; and b) a means for recovering said antibody from said mammal. In a still further embodiment, a kit is provided for producing an antibody to a parathyroid hormone (PTH) or a PTH peptide, which kit comprises: a) a PTH protein or peptide from between PTH₁₋₈₄ and PTH₃₅₋₈₄; b) means for introducing said PTH protein or peptide from between PTH₁₋₈₄ and PTH₃₅₋₈₄ to a mammal in an amount sufficient to produce an antibody to said PTH protein or peptide; c) means for recovering said antibody from said mammal; and c) another specific PTH peptide.

An isolated PTH peptide in such kits can be any of the variety of PTH peptides as described herein. Frequently, the PTH peptides are conjugated to a carrier to enhance the PTH peptide's immunogenicity, e.g., a carrier protein, which may together form a fusion protein. For example, such PTH peptide is selected from the group consisting of PTH₈₁₋₈₄, PTH₈₀₋₈₄, PTH₇₉₋₈₄, PTH₇₈₋₈₄, PTH₇₇₋₈₄, PTH₇₆₋₈₄, PTH₇₅₋₈₄, PTH₇₄₋₈₄, PTH₇₃₋₈₄, PTH₇₂₋₈₄, PTH₇₁₋₈₄ or PTH₇₀₋₈₄.

The presently contemplated kits may also provide an immunogen comprising a PTH peptide as described herein, together with an immune response potentiator. On occasion, the immune response potentiator is selected from the group consisting of Bacille Calmette-Guerin (BCG), Corynebacterium Parvum, Brucella abortus extract, glucan, levamisole, tilorone, an enzyme and a non-virulent virus.

The present disclosure further provides methods for producing an antibody to a parathyroid hormone (PTH) or a PTH peptide. In one embodiment, such method comprises: a) introducing an isolated PTH peptide to a mammal in an amount sufficient to produce an antibody to said PTH peptide; and b) recovering said antibody from said mammal. Another frequent method for producing an antibody to a parathyroid hormone (PTH) or a PTH peptide comprises: a) introducing a MAP to a mammal in an amount sufficient to produce an antibody to a PTH peptide comprised in said MAP; and b) recovering said antibody from said mammal. In one aspect, the present disclosure provides antibodies to a PTH or a PTH peptide produced by these methods. In a related embodiment, a method is provided for producing an antibody to a parathyroid hormone (PTH) or a PTH peptide, which method comprises: a) introducing a PTH protein or peptide from between PTH₁₋₈₄ and PTH₃₅₋₈₄ to a mammal in an amount sufficient to produce an antibody to said PTH protein or peptide; b) recovering said antibody from said mammal; and c) affinity purifying a PTH antibody that specifically binds to an epitope comprised in a PTH peptide using said PTH peptide. In a further embodiment, the present disclosure provides an antibody to a PTH or a PTH peptide produced by such methods.

### V. BRIEF DESCRIPTION OF THE DRAWINGS

FIG. 1 is a diagrammatic view of whole human PTH (SEQ ID NO: 1).

FIGS. 2A and 2B are diagrammatic views of wPTH assays using the present antibodies as capture and tracer elements. FIG. 2A refers to the ELISA assay format, whereas FIG. 2B refers to the IRMA assay format.

FIGS. 3A and 3B depict different epitope specificities of the antibodies used in the whole rPTH and total rPTH assays. FIG. 3A shows that rPTH (1-34) and hPTH (1-34) strongly inhibited the wPTH assay, while hPTH (2-38) demonstrated moderate inhibition, indicating that the epitope of the detection antibody used in the whole rPTH assay was part of the rPTH (1-34), and the first amino acid was involved in binding to the detection antibody. The detection antibody in this assay did not react with rPTH (7-34), indicating that the rat whole PTH assay detects only the rPTH with an intact N-terminus. In contrast, FIG. 3B demonstrates that the total rPTH assay was not inhibited by hPTH (1-34), and the epitope of the detection antibody used in the total rPTH assay was part of rPTH (7-34) rather than rPTH (1-6).

FIG. 4 depicts epitope specificity of the antibody used in the whole rPTH assay (wPTH). The assay did not detect any rPTH (7-84) (closed triangles) or rPTH (39-84) (open triangles), indicating that the rat whole PTH ELISA detected only rPTH (1-84) (closed squares). Standard Intact Scantibodies (closed circles): rat PTH (1-84) standard from Scantibodies; rPTH PTH (1-84) home (closed squares): rat PTH (1-84) standard from Dr. Pierre D'Amour's lab; rPTH PTH (7-84) home (closed triangles): rat PTH (7-84) standard from Dr. Pierre D'Amour's lab; rat PTH (39-84): rat PTH (39-84) standard.

FIG. 5 depicts epitope specificity of the antibody used in the total rPTH assay (tPTH). The assay detected both rPTH (1-84) (closed squares) and rat PTH (7-84) (closed triangles). However, the assay did not detect any rPTH (39-84) (open triangles). Standard Intact Scantibodies (closed circles): rat PTH (1-84) standard from Scantibodies; rPTH PTH (1-84) home (closed squares): rat PTH (1-84) standard from Dr. Pierre D'Amour's lab; rPTH PTH (7-84) home (closed triangles): rat PTH (7-84) standard from Dr. Pierre D'Amour's lab; rat PTH (39-84): rat PTH (39-84) standard.

FIG. 6 depicts epitope specificity of the antibody used in the C-terminal rPTH assay (cPTH). The assay detected all of rPTH (1-84) (closed squares), rPTH (7-84) (closed triangles) and rPTH (39-84) (open triangles). However, the rat PTH (39-84) signal appeared to be more potent than the signals from rPTH (1-84) and rPTH (7-84). Standard Intact Scantibodies (closed circles): rat PTH (1-84) standard from Scantibodies; rPTH PTH (1-84) home (closed squares): rat PTH (1-84) standard from Dr. Pierre D'Amour's lab; rPTH PTH (7-84) home (closed triangles): rat PTH (7-84) standard from Dr. Pierre D'Amour's lab; rat PTH (39-84): rat PTH (39-84) standard.

FIG. 7 depicts the PTH composition of rat serum in male rats. Rat serum pooled from normal 75-day male rats was fractionated on a HPLC reverse-phased column. The dotted peaks on the far left are cPTH; those in the middle represent PTH (7-84) and other N-terminus-truncated large fragments; and the large peak on the far right is PTH (1-84).

FIG. 8 depicts the PTH composition of rat serum in female rats. Rat serum pooled from normal 75-day female rats was fractionated on a HPLC reverse-phased column. The dotted peaks on the far left are cPTH; those in the middle represent PTH (7-84) and other N-terminus-truncated large fragments; and the large peak on the far right is PTH (1-84).

FIG. 9 depicts an HPLC study of serum collected from rats maintained on a normal calcium diet. The dotted peaks on the far left are cPTH; those in the middle represent PTH (7-84) and other N-terminus-truncated large fragments; and the large peak on the far right is PTH (1-84).

FIG. 10 depict an HPLC study of serum collected from rats maintained on a low calcium diet. The dotted peaks on the far left are cPTH; those in the middle represent PTH (7-84) and other N-terminus-truncated large fragments; and the large peak on the far right is PTH (1-84).

FIG. 11 depicts an HPLC study of serum collected from rats maintained on a vitamin D deficient diet. The dotted peaks on the far left are cPTH; those in the middle represent PTH (7-84) and other N-terminus-truncated large fragments; and the large peak on the far right is PTH (1-84).

FIG. 12 depicts an HPLC study of rat serum collected from rats with partially removed kidneys. The dotted peaks on the far left are cPTH; those in the middle represent PTH (7-84) and other N-terminus-truncated large fragments; and the large peak on the far right is PTH (1-84).

### VI. DETAILED DESCRIPTION OF THE INVENTION

### A. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “antibody” is used in the broadest sense. Therefore, an “antibody” can be naturally occurring or man-made such as monoclonal antibodies produced by conventional hybridoma technology and/or a functional fragment thereof. Antibodies of the present invention comprise monoclonal and polyclonal antibodies as well as fragments containing the antigen-binding domain and/or one or more complementarity determining regions of these antibodies.

As used herein, “monoclonal antibody” refers to an antibody obtained from a population of substantially homogeneous antibodies, i.e., the antibodies comprising the population are identical except for possible naturally occurring mutations that are present in minor amounts. As used herein, a “monoclonal antibody” further refers to functional fragments of monoclonal antibodies.

As used herein, “mammal” refers to any of the mammalian class of species. Frequently, the term “mammal,” as used herein, refers to humans, human subjects or human patients.

As used herein, “whole parathyroid hormone (PTH)” or “wPTH” refers to the complete molecule of PTH. This term is not species-specific unless otherwise designated. For purposes herein, the name “parathyroid hormone (PTH)” is used herein, although all other names are contemplated. It is intended to encompass whole PTH with conservative amino acid substitutions that do not substantially alter its biological activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al., MOLECULAR BIOLOGY OF THE GENE, 4th Edition, 1987, The Bejamin/Cummings Pub. Co., p. 224).

As used herein, “parathyroid hormone (PTH) agonist,” “cyclase activating PTH” or “CAP” refers to the complete molecule of PTH or a fragment, derivative or analog thereof that stimulates osteoclasts formation and bone turnover to increase blood calcium levels. PTH agonist further refers to peptides which have PTH agonist properties. Other names of PTH include parathormone and parathyrin. For purposes herein, the name “parathyroid hormone (PTH)” is used herein, although all other names are contemplated. It is intended to encompass PTH agonist with conservative amino acid substitutions that do not substantially alter its biological activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al., MOLECULAR BIOLOGY OF THE GENE, 4th Edition, 1987, The Bejamin/Cummings Pub. co., p. 224). PTH agonist assay values may be obtained by measuring a sample with a Scantibodies Whole PTH Assay or a Scantibodies CAP Assay or a 3rd generation PTH Assay or a Nichols BioIntact PTH assay or an Immutopics Human Bioactive PTH assay.

As used herein, the term “total PTH” refers to a total accounting of whole PTH levels in addition to non-whole PTH fragment(s) levels. Moreover, this term is not species-specific unless otherwise designated.

As used herein, the term “non-whole PTH” refers to a PTH fragment generated by a cleavage near the N-terminus and having an intact C-terminus. In one embodiment, the “non-whole PTH” refers to a group of PTH fragments, PTH₄₋₈₄, PTH₅₋₈₄, PTH₆₋₈₄, PTH₇₋₈₄, PTH₈₋₈₄, PTH₉₋₈₄, PTH₁₀₋₈₄, PTH₁₁₋₈₄, PTH₁₂₋₈₄, PTH₁₃₋₈₄, PTH₁₄₋₈₄, and PTH₁₅₋₈₄.

As used herein, the term “PIN” refers to PTH fragments that have PTH antagonistic or inhibiting properties. Therefore, although occasionally of concurrent scope, a reference to PTH fragments, as provided herein, is not intended to be limited to PIN. In one embodiment, the term “PIN” refers to “non-whole PTH.”

As used herein, a “PTH fragment” is a PTH peptide that comprises a non-whole contiguous portion of an entire PTH protein. A reference to a PTH fragment as herein includes C-terminal, N-terminal, mid-terminal fragments and PIN, unless otherwise indicated. Moreover, this term is not species-specific unless otherwise designated.

As used herein, the term “N-terminal” or “N-terminus” refers to the amino terminus of a PTH polypeptide having a free amino group. With reference to a PTH fragment, an “N-terminal PTH fragment” refers to a non-whole contiguous portion of PTH having an intact N-terminus. An “intact N-terminal” or “N-terminus” as used herein refers to PTH or a PTH fragment having an intact 1st position of PTH₁₋₈₄. This first position is also referred to herein as an “original N-terminal” or an “original N-terminus.”

As used herein, the term “C-terminal” or “C-terminus” refers to the carboxyl terminus of a PTH polypeptide having a free carboxyl group and is shorter than a “non-whole PTH.” With reference to a PTH fragment, a “C-terminal PTH fragment” refers to a non-whole contiguous portion of PTH having an intact C-terminus. An “intact C-terminal” or “intact C-terminus” as used herein refers to PTH or a PTH fragment having an intact 84th position of PTH₁₋₈₄ or an intact 86th position of PTH₁₋₈₆. This 84th or 86th position is also referred to herein as an “original C-terminal” or an “original C-terminus.” In one embodiment, the “C-terminal PTH fragment” refers to a group of PTH fragments, PTH₃₅₋₈₄ to PTH₆₀₋₈₄.

As used herein, the term “mid-terminal PTH fragment” refers to a non-whole contiguous portion of PTH having neither an intact N-terminus nor an intact C-terminus. These types of PTH fragments may also be referred to herein as “mid-terminus fragments.”

As used herein, “treatment” means any manner in which the symptoms of a condition, disorder or disease are ameliorated or otherwise beneficially altered. Treatment also encompasses any pharmaceutical use of the compositions herein.

As used herein, “disease or disorder” refers to a pathological condition in an organism resulting from, e.g., infection or genetic defect, and characterized by identifiable symptoms.

As used herein, “high bone turnover” refers to the bone turnover rate as being above a normal bone turnover rate in a subject and is one of the symptoms manifested in subjects having hyperparathyroidism. While not bound by theory, a subject afflicted with severe hyperparathyroidism has a higher bone turnover rate than the same subject afflicted with mild hyperparathyroidism, however, both having a high bone turnover rate as compared with a normal subject and a subject afflicted with adynamic bone disease.

As used herein, the term “subject” is not limited to a specific species or sample type. For example, the term “subject” may refer to a patient, and frequently a human patient. However, this term is not limited to humans and thus encompasses a variety of mammalian species.

As used herein, “afflicted” as it relates to a disease or disorder refers to a subject having or directly affected by the designated disease or disorder.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein the term “physiological level of whole PTH” refers generally to the average concentration of whole PTH present in a mammal, e.g., a human, expressed in pmol/L, or another suitable measurement unit (e.g., pg/ml). See, e.g., Woodhead, J. S., Clin. Biochem. 23, 17 (1990). In one aspect, the physiological range of whole PTH ranges between about 0.2 pmol/L to about 4 pmol/L, or about 2 pg/ml to about 40 pg/ml. On occasion, the physiological range of whole PTH can range between about 7 pg/ml to about 39 pg/ml. Although specific ranges are described herein as representative of a physiological range, one of skill in the art would understand that the physiological level of whole PTH may lie outside of the presently disclosed ranges in certain subjects. Nevertheless, the compositions and methods provided herein are useful to detect discreet concentrations of whole PTH and have sensitivities within the physiological range as provided herein.

As used herein, the term “specifically binds” refers to the specificity of an antibody such that it preferentially binds to a defined target. Recognition by an antibody of a particular target in the presence of other potential targets is one characteristic of such binding. Specific binding of the presently contemplated antibodies to particular PTH targets is measured through known methods utilizing the tools provided herein. Antibodies or antibody fragments that specifically bind to a moiety having a particular region, sequence or epitope generally contain a specificity such that only a low percentage of such antibodies or antibody fragments would bind to an interfering moiety not having the particular region, sequence or epitope. This percentage generally lies within the acceptable cross reactivity percentage with interfering moieties of assays utilizing antibodies directed to detecting a specific target. Frequently, antibodies or antibody fragments of the present disclosure that specifically bind a PTH or a PTH peptide having an intact C-terminus bind less than about 10% of an interfering moiety, although lower percentages are clearly contemplated and preferred. For example, antibodies or antibody fragments of the present disclosure bind about 9%, about 8%, about 7%, about 6%, about 5%, about 4%, about 3%, about 2%, about 1%, and about 1% or less of an interfering moiety. Less occasionally, antibodies or antibody fragments of the present disclosure bind less than about 30%, or less than about 25%, or less than about 20%, or less than about 15% of an interfering moiety. Although not bound by theory, as contemplated herein, an interfering moiety may comprise a PTH or a PTH peptide missing one or more C-terminal amino acids.

As used herein, “stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Current Protocols in Molecular Biology (Ausubel et al. eds., Wiley Interscience Publishers, 1995); Molecular Cloning: A Laboratory Manual (J. Sambrook, E. Fritsch, T. Maniatis eds., Cold Spring Harbor Laboratory Press, 2d ed. 1989); Wood et al., Proc. Natl. Acad. Sci. USA, 82:1585-1588 (1985).

As used herein the term “isolated” refers to material removed from its original environment, and is altered from its natural state. For example, an isolated polypeptide could be coupled to a carrier, and still be “isolated” because that polypeptide is not in its original environment.

The present disclosure encompasses antigens, antibodies and methods of producing antibodies that have a particular specificity to target proteins and/or peptides which contain a specific amino acid residue or multiple amino acid residues, in a series or otherwise. The specific amino acid residue(s) may be located in the N-terminal region of a proteins or peptide or in the C-terminal region. Moreover the specific amino acid residue(s) may be located in a region between the N-terminal and C-terminal regions of a protein or peptide. Occasionally, when there is more than one specific amino acid residue, such residues may be dispersed in any one or more of the N-terminal, C-terminal, between these two regions, and/or in all of these regions.

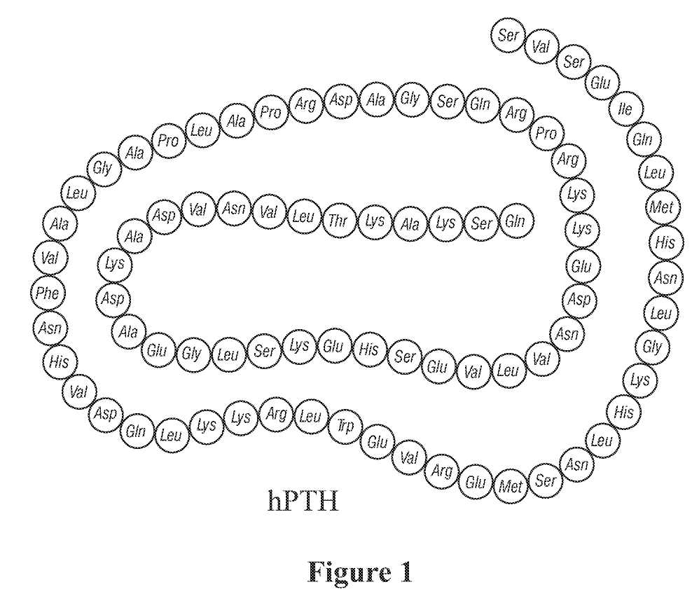

In disclosing the present invention, one should remember that there are a number of closely analogous, species dependent forms of PTH. The amino acid sequence of hPTH is shown in FIG. 1. However, for rat PTH, mouse PTH, bovine PTH, canine PTH, horse PTH or porcine PTH, for example, one finds the substitutions at some of the amino acids in the hPTH sequence. For the purposes of the present invention, one can use interchangeably antibodies or antibody fragments to forms of these PTHs, although it is preferred to use an antibody with specificity for PTH having a sequence matching the species in which the PTH measurements are made.

### B. Parathyroid Hormone Fragments

In general, a PTH fragment of the present invention comprises a non-whole contiguous portion of PTH having an amino acid sequence as set forth in SEQ ID NOs: 1 2, 3, 4, 5, 6, and/or 7 (PTH₁₋₈₄ or PTH₁₋₈₆), or a nucleic acid encoding said portion of PTH. A PTH fragment may have the following characteristics: a) the N-terminal amino acid residue of said PTH fragment starts at any position spanning position 1 through position 80 of said PTH₁₋₈₄; b) the C-terminal amino acid residue of said PTH fragment ends at any position spanning position 4 through position 84 of said PTH₁₋₈₄; and c) said PTH fragment has a minimal length of three amino acid residues. Preferably, the PTH fragment is in the form of a pharmaceutical composition.

PTH fragments of the present invention are organized into three categories: N-terminal, C-terminal, and mid-terminal PTH fragments. As further described herein, N-terminal fragments comprise a non-whole contiguous portion of PTH having an intact N-terminus, but not an intact original C-terminus. As also described herein, C-terminal fragments comprise a non-whole contiguous portion of PTH having an intact C-terminus, but not an intact original N-terminus. Moreover, as further described herein, mid-terminal fragments comprise a non-whole contiguous portion of PTH having neither an intact original C-terminus, nor intact original N-terminus. All mammalian sources/sequences of PTH are contemplated.

In one embodiment, C-terminal PTH fragments comprise a subset of cyclase inactive PTH. However, in light of the present description, a variety of other PTH fragments are contemplated, ascertainable and useful in the present compositions, kits and methods. Importantly, PTH₃₅₋₈₄ represents a member of the group of C-terminal PTH fragments currently contemplated.

In one embodiment, the N-terminal amino acid residue of the non-whole PTH fragment starts at any defined position spanning position 4 through position 34 of said PTH₁₋₈₄. The C-terminal amino acid residue of said non-whole PTH fragment ends at the 84th position of said PTH₁₋₈₄ or the 86th position of said PTH₁₋₈₆. Therefore, for example, fragments ranging from PTH₄₋₈₄ to PTH₁₅₋₈₄ are included as non-whole PTH fragments.

In a specific embodiment, the C-terminal PTH fragment is a protein or a peptide, or a nucleic acid encoding said protein or peptide, selected from the group consisting of PTH₃₅₋₈₄, PTH₃₆₋₈₄, PTH₃₇₋₈₄, PTH₃₈₋₈₄, PTH₃₉₋₈₄, PTH₀₋₈₄, PTH₄₁₋₈₄, PTH₄₂₋₈₄, PTH₄₃₋₈₄, PTH₄₄₋₈₄, PTH₄₅₋₈₄, PTH₄₆₋₈₄, PTH₄₇₋₈₄, PTH₄₈₋₈₄, PTH₄₉₋₈₄, PTH₅₀₋₈₄, PTH₅₁₋₈₄, PTH₅₂₋₈₄, PTH₅₃₋₈₄, PTH₅₄₋₈₄, PTH₅₅₋₈₄, PTH₅₆₋₈₄, PTH₅₇₋₈₄, PTH₅₈₋₈₄, PTH₅₉₋₈₄ and PTH₆₀₋₈₄.

In another preferred embodiment, a mid-terminal PTH fragment comprises, or an antibody specifically binds, a PTH peptide fragment selected from the group consisting of: PTH₄₀₋₆₀, PTH₃₅₋₄₀, PTH₅₃₋₇₃, and PTH₃₅₋₇₄, or a combination of two or more from this group. In a more preferred embodiment, a mid-terminal PTH fragment comprises, or an antibody specifically binds, the PTH₄₀₋₆₀ fragment. In a particularly preferred embodiment, a PTH peptide fragment comprises a PTH fragment present and detectable in nature.

The PTH fragment can have any suitable length and may have PTH agonizing or antagonizing activity, although PTH agonizing or antagonizing activity is not required of the present PTH fragments. For example, the PTH fragment can have a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 or 83 amino acid residues.

### C. PTH Ratios

### Whole PTH and PTH Fragments

An important discovery leading to the present invention is that adynamic bone loses its capacity to buffer calcium and phosphate as the bones are shut down. In subjects afflicted with such conditions, they are unable to effectively buffer calcium as it enters their bodies through their diet. This calcium enters the blood stream and is thereafter shuttled to the soft tissues. The parathyroid gland is particularly subject to, and detrimentally affected by, this influx of calcium and thereby produces PTH fragments rather than, or in addition to, the active form of PTH. See, e.g., Mayer GP, et al., Endocrinology 104: 1778-1784 (1979); D'Amour P, et al., J. Clin. Endocrinol. Metab. 74: 525-532 (1992); D'Amour P, et al., J. Bone Miner. Res. 11: 1075-1085 (1996); Cardinal, H., et al., J. Clin. Endocrinol. Metab. 83: 3839-44 (1998). Accordingly, in subjects with adynamic bone, the concentration and production of PTH fragments is increased. In light of this and other related information, the measurement of PTH fragment levels, and particularly in conjunction with the measurement of whole PTH, can be used effectively to differentiate subjects having adynamic bone versus those having normal bone and high bone turnover rates.

The present disclosure includes these findings in the presentation of peptides, antibodies, methods and kits for the measurement of PTH levels. In one preferred embodiment, the present methods utilize a ratio of C-terminal PTH to whole PTH (i.e., cPTH/wPTH ratio). In another preferred embodiment, the present methods utilize a ratio of C-terminal PTH to total PTH (i.e., cPTH/total PTH ratio), wherein the total PTH level comprises whole PTH plus PTH fragments, such as other PTH fragments described herein. Until recently, the predictive and therapeutic benefits of PTH ratios remained largely unrecognized. See, e.g., Martine-Esther Cohen Solal, et al., J. Clin. Endocrinol. Metab. 73: 516-524 (1991) (concluding that the measurement of whole PTH is “superior to C-terminal and midregion assays for the prediction of histological type bone diseases.”).

In one embodiment, a C-terminal PTH assay is utilized wherein an antibody specific for PTH₄₀₋₆₀ is utilized in addition to an antibody specific for PTH₇₅₋₈₄ in addition to other antibodies to determine a total PTH level.

There is a compelling need to be able to non-invasively separate the dialysis patients with ADN from those suffering from high bone turnover to avoid over treatment of ADN dialysis patients. Over treatment of dialysis patients with ADN is a frequent occurrence under presently utilized methods. For example, calcium based phosphate binders such as Zemplar® and Calcijex® (Abbott Laboratories), for example, have been used to treat dialysis patients. Under the treatment protocols utilized and recommended, these patients are at a great risk of over treatment due to inaccurate measurement of PTH levels (including wPTH and PTH fragment levels). For example, the proportion of dialysis patients treated with calcium based phosphate binders that become afflicted with ADN rose sharply during the time spanning 1995 to 2000 from 12% to 48% of such patients. See, e.g., Malluche, H. H., The Importance of Bone Health in ESRD: Out of the Frying Pan, Into the Fire?, World Congress on Nephrology, Berlin, Germany (June 2003) (based on unpublished data). It is postulated that this increase is due, in large part, to over treatment of dialysis patients; and this over treatment of dialysis patients is due, in turn, to ineffective PTH level monitoring (including whole PTH and PTH fragment levels). Moreover, K/DOQI recommends whole PTH as the only marker useful for separating ADN from HBT dialysis patients. See K/DOQI Clinical Practice Guidelines for Bone Metabolism and Disease in Chronic Kidney Disease, Draft Guideline Statements and Treatment Algorithms (February 2003). However, whole PTH levels fail to consistently separate ADN from HBT dialysis patients. See Qi, Q, et al., Am. J. Kidney Dis. 26:622-31 (1995); Quarles, L D, et al., J. Clin. Endocrinol. Metab. 75:145-150 (1992). It is recognized herein that such guidelines would appear to propagate the over treatment phenomenon. Accordingly, the present compositions and methods is aimed at improving the effectiveness of separating ADN from HBT dialysis patients via PTH ratio results, rather than via measurement of whole PTH levels alone, and their use for routine clinical management of chronic renal disease, osteoporosis, and/or dialysis patients.

### D. PTH Assay Locations

The presently contemplated methods may be performed in a variety of settings and by a variety of entities. However, in general, the present methods and materials may be made available in a health care setting. Frequently, the present methods, e.g., determining and monitoring of PTH levels and ratios as described herein, are utilized in the clinical management of disease or disorders in a subject by a care provider or clinical laboratory. A health care setting, as used herein, includes clinical laboratories, doctor's offices, hospitals, health management organization facilities, and outpatient care facilities, amongst a variety of other nontraditional settings useful for the delivery of care and subject testing.

### E. PTH Sequences

The present disclosure contemplates the use of parathyroid hormone peptides, peptide fragments, polynucleotides encoding whole PTH or PTH fragment peptides, and antibodies that specifically bind whole PTH and/or PTH fragments derived from a variety of mammalian sources. See, e.g., Caetano, A. R., et al., Equus Genome Res. 9(12): 1239-1249 (1999) (horse), U.S. Patent Application Publication US 2002/0110871 A1 (rat, mouse, bovine, canine, porcine), U.S. patent application Ser. Nos. 09/344,639 and 09/231,422 (human). By way of nonlimiting example, PTH derived from the following sources and having the following peptide sequences are contemplated herein:

Human PTH₁₋₈₄ (SEQ ID NO: 1):
SER-VAL-SER-GLU-ILE-GLN-LEU-MET-HIS-ASN-LEU-GLY-
LYS-HIS-LEU-ASN-SER-MET-GLU-ARG-VAL-GLU-TRP-LEU-
ARG-LYS-LYS-LEU-GLN-ASP-VAL-HIS-ASN-PHE-VAL-ALA-
LEU-GLY-ALA-PRO-LEU-ALA-PRO-ARG-ASP-ALA-GLY-SER-
GLN-ARG-PRO-ARG-LYS-LYS-GLU-ASP-ASN-VAL-LEU-VAL-
GLU-SER-HIS-GLU-LYS-SER-LEU-GLY-GLU-ALA-ASP-LYS-
ALA-ASP-VAL-ASN-VAL-LEU-THR-LYS-ALA-LYS-SER-GLN.
Rat PTH₁₋₈₄ (SEQ ID NO: 2):
ALA-VAL-SER-GLU-ILE-GLN-LEU-MET-HIS-ASN-LEU-GLY-
LYS-HIS-LEU-ALA-SER-VAL-GLU-ARG-MET-GLN-TRP-LEU-
ARG-LYS-LYS-LEU-GLN-ASP-VAL-HIS-ASN-PHE-VAL-SER-
LEU-GLY-VAL-GLN-MET-ALA-ALA-ARG-GLU-GLY-SER-TYR-
GLN-ARG-PRO-THR-LYS-LYS-GLU-ASP-ASN-VAL-LEU-VAL-
ASP-GLY-ASN-SER-LYS-SER-LEU-GLY-GLU-GLY-ASP-LYS-
ALA-ASP-VAL-ASP-VAL-LEU-VAL-LYS-ALA-LYS-SER-GLN.
Mouse PTH₁₋₈₄ (SEQ ID NO: 3):
ALA-VAL-SER-GLU-ILE-GLN-LEU-MET-HIS-ASN-LEU-GLY-
LYS-HIS-LEU-ALA-SER-VAL-GLU-ARG-MET-GLN-TRP-LEU-
ARG-ARG-LYS-LEU-GLN-ASP-MET-HIS-ASN-PHE-VAL-SER-
LEU-GLY-VAL-GLN-MET-ALA-ALA-ARG-ASP-GLY-SER-HIS-
GLN-LYS-PRO-THR-LYS-LYS-GLU-GLU-ASN-VAL-LEU-VAL-
ASP-GLY-ASN-PRO-LYS-SER-LEU-GLY-GLU-GLY-ASP-LYS-
ALA-ASP-VAL-ASP-VAL-LEU-VAL-LYS-SER-LYS-SER-GLN.
Bovine PTH₁₋₈₄ (SEQ ID NO: 4):
ALA-VAL-SER-GLU-ILE-GLN-PHE-MET-HIS-ASN-LEU-GLY-
LYS-HIS-LEU-SER-SER-MET-GLU-ARG-VAL-GLU-TRP-LEU-
ARG-LYS-LYS-LEU-GLN-ASP-VAL-HIS-ASN-PHE-VAL-ALA-
LEU-GLY-ALA-SER-ILE-ALA-TYR-ARG-ASP-GLY-SER-SER-
GLN-ARG-PRO-ARG-LYS-LYS-GLU-ASP-ASN-VAL-LEU-VAL-
GLU-SER-HIS-GLN-LYS-SER-LEU-GLY-GLU-ALA-ASP-LYS-
ALA-ASP-VAL-ASP-VAL-LEU-ILE-LYS-ALA-LYS-PRO-GLN.
Canine PTH₁₋₈₄ (SEQ ID NO: 5):
SER-VAL-SER-GLU-ILE-GLN-PHE-MET-HIS-ASN-LEU-GLY-
LYS-HIS-LEU-SER-SER-MET-GLU-ARG-VAL-GLU-TRP-LEU-
ARG-LYS-LYS-LEU-GLN-ASP-VAL-HIS-ASN-PHE-VAL-ALA-
LEU-GLY-ALA-PRO-ILE-ALA-HIS-ARG-ASP-GLY-SER-SER-
GLN-ARG-PRO-LEU-LYS-LYS-GLU-ASP-ASN-VAL-LEU-VAL-
GLU-SER-TYR-GLN-LYS-SER-LEU-GLY-GLU-ALA-ASP-LYS-
ALA-ASP-VAL-ASP-VAL-LEU-THR-LYS-ALA-LYS-SER-GLN.
Porcine PTH₁₋₈₄ (SEQ ID NO: 6):
SER-VAL-SER-GLU-ILE-GLN-PHE-MET-HIS-ASN-LEU-GLY-
LYS-HIS-LEU-SER-SER-LEU-GLU-ARG-VAL-GLU-TRP-LEU-
ARG-LYS-LYS-LEU-GLN-ASP-VAL-HIS-ASN-PHE-VAL-ALA-
LEU-GLY-ALA-SER-ILE-VAL-HIS-ARG-ASP-GLY-GLY-SER-
GLN-ARG-PRO-ARG-LYS-LYS-GLU-ASP-ASN-VAL-LEU-VAL-
GLU-SER-HIS-GLN-LYS-SER-LEU-GLY-GLU-ALA-ASP-LYS-
ALA-ALA-VAL-ASP-VAL-LEU-ILE-LYS-ALA-LYS-PRO-GLN.
Horse PTH₁₋₈₆ (SEQ ID NO: 7):
LYS-ARG-SER-VAL-SER-GLU-ILE-GLN-LEU-MET-HIS-ASN-
LEU-GLY-LYS-HIS-LEU-ASN-SER-VAL-GLU-ARG-VAL-GLU-
TRP-LEU-ARG-LYS-LYS-LEU-GLN-ASP-VAL-HIS-ASN-PHE-
ILE-ALA-LEU-GLY-ALA-PRO-ILE-PHE-HIS-ARG-ASP-GLY-
GLY-SER-GLN-ARG-PRO-ARG-LYS-LYS-GLU-ASP-ASN-VAL-
LEU-ILE-GLU-SER-HIS-GLN-XXX-SER-LEU-GLY-GLU-ALA-
ASP-LYS-ALA-ASP-VAL-ASP-VAL-LEU-SER-LYS-THR-LYS-
SER-GLN.

### II. EXEMPLARY MODES FOR CARRYING OUT THE INVENTION

In disclosing the present invention, one should remember that there are a number of closely analogous, species dependent forms of PTH (see above). The amino acid sequence of hPTH is shown in FIG. 1. However, for rat PTH, bovine PTH, or porcine PTH, for example, one finds the substitutions at some of the amino acids in the hPTH sequence (see, e.g., SEQ ID NOs: 1-7). For the purposes of the present invention, one can use interchangeably antibodies or antibody fragments to forms of these PTHs, although it is preferred to use an antibody with specificity for PTH having a sequence matching the species in which the PTH measurements are made.

Any suitable whole PTH assays and total PTH assays can be used in the present invention. Exemplary whole PTH assays, initial whole PTH sequence peptides, initial sequence whole PTH antibodies, and total PTH assays in U.S. Pat. Nos. 6,689,566 and 6,743,590, and in the co-pending U.S. application Ser. No. 10/617,489, 11/799,726 and 11/894,367 can be used in the present invention.

A. C-Terminal Sequence PTH Antibody

To create an affinity-purified anti-(75-84) PTH antibody, one first uses a selected C-terminal PTH sequence peptide as described above as part of an immunogen for injection into a host, e.g., a goat. In another embodiment, the immunogen comprises whole PTH peptide, e.g., PTH₁₋₈₄ or PTH₁₋₈₆. The peptide can be used either by itself as an injectable immunogen, incorporated into a non PTH peptide having a molecular weight, typically, of between about 5,000 and 10,000,000. The immunogen is mixed with an equal volume of Freund's complete adjuvant which is a mixture of light mineral oil, Arlacel detergent, and inactivated mycobacterium tuberculosis bacilli. The resulting mixture is homogenized to produce an aqueous/oil emulsion which is injected into the animal (typically a goat) for the primary immunization. The immunogen dose is approximately 50-400 micrograms. The goats are injected monthly with the same dose of immunogen complex except no mycobacterium tuberculosis bacilli is used in these subsequent injections. The goats are bled monthly, approximately three months after the primary immunization. The serum (or antiserum) is derived from each bleeding by separating the red blood cells from the blood by centrifugation and removing the antiserum which contains (75-84) PTH antibodies. In another embodiment, the antiserum is removed which contains (75-84) PTH antibodies in addition to other PTH antibodies, e.g., whole PTH antibodies.

To purify the antiserum for the desired (75-84) PTH antibody, one packs a separation column with the C-terminal PTH sequence peptide bound beads described above, washes the column and equilibrates it with 0.01 M phosphate buffered saline (PBS). The antiserum is loaded onto the column and washed with 0.01 M PBS in order to remove antibodies without the (75-84) PTH specificity. The bound specific goat anti-(75-84) PTH polyclonal antibody is eluted from the solid phase PTH₇₅₋₈₄ in the column by passing an elution solution of 0.1 M glycine hydrochloride buffer, pH 2.5 through the column. The eluted polyclonal antibody is neutralized after it leaves the column with either the addition of 1.0 M phosphate buffer, pH 7.5 or by a buffer exchange with 0.01 M PBS, as is known to those of skill in the art. The polyclonal antibody is stored at 2-8 degrees centigrade.

One of skill in the art would understand that there are acceptable variations in the above practices. See, e.g., Harlow E, Lane D: Antibodies: A laboratory manual. Cold Spring Harbor Laboratory Press, Cold Spring Harbor, N.Y., 1988; Kohler & Milstein, Nature, 256: 495-7 (1975). While not bound by theory, the above practices are suitable for production of other PTH C-terminal antibodies using selected C-terminal PTH sequence peptides as described herein. For example, PTH peptides such as PTH₈₁₋₈₄, PTH₈₀₋₈₄, PTH₇₉₋₈₄, PTH₇₈₋₈₄, PTH₇₇₋₈₄, PTH₇₆₋₈₄, PTH₇₄₋₈₄, PTH₇₃₋₈₄, PTH₇₂₋₈₄, PTH₇₁₋₈₄ or PTH₇₀₋₈₄, and other C-terminal PTH peptides as described above.

In a particularly preferred embodiment, C-terminal PTH antibodies successfully distinguish between C-terminal peptides and both initial sequence and mid-terminus PTH peptides, such that they specifically bind C-terminal PTH peptides.

B. Mid-Terminus Sequence PTH Antibody

To create an affinity-purified anti-(40-60) PTH antibody, one first uses a selected mid-terminus PTH sequence peptide as described above as part of an immunogen for injection into a host, e.g., a goat. In another embodiment, the immunogen comprises whole PTH peptide, e.g., PTH₁₋₈₄. The peptide can be used either by itself as an injectable immunogen, incorporated into a non PTH peptide having a molecular weight, typically, of between about 5,000 and 10,000,000. The immunogen is mixed with an equal volume of Freund's complete adjuvant which is a mixture of light mineral oil, Arlacel detergent, and inactivated mycobacterium tuberculosis bacilli. The resulting mixture is homogenized to produce an aqueous/oil emulsion which is injected into the animal (typically a goat) for the primary immunization. The immunogen dose is approximately 50-400 micrograms. The goats are injected monthly with the same dose of immunogen complex except no mycobacterium tuberculosis bacilli is used in these subsequent injections. The goats are bled monthly, approximately three months after the primary immunization. The serum (or antiserum) is derived from each bleeding by separating the red blood cells from the blood by centrifugation and removing the antiserum which contains (40-60) PTH antibodies. In another embodiment, the antiserum is removed which contains (40-60) PTH antibodies in addition to other PTH antibodies, e.g., whole PTH antibodies.

To purify the antiserum for the desired (40-60) PTH antibody, one packs a separation column with the mid-terminus PTH sequence peptide bound beads described above, washes the column and equilibrates it with 0.01 M phosphate buffered saline (PBS). The antiserum is loaded onto the column and washed with 0.01 M PBS in order to remove antibodies without the (40-60) PTH specificity. The bound specific goat anti-(40-60) PTH polyclonal antibody is eluted from the solid phase PTH₄₀₋₆₀ in the column by passing an elution solution of 0.1 M glycine hydrochloride buffer, pH 2.5 through the column. The eluted polyclonal antibody is neutralized after it leaves the column with either the addition of 1.0 M phosphate buffer, pH 7.5 or by a buffer exchange with 0.01 M PBS, as is known to those of skill in the art. The polyclonal antibody is stored at 2-8 degrees centigrade.

One of skill in the art would understand that there are acceptable variations in the above practices. See, e.g., Harlow E, Lane D: Antibodies: A laboratory manual. Cold Spring Harbor Laboratory Press, Cold Spring Harbor, N.Y., 1988; Kohler & Milstein, Nature, 256: 495-7 (1975). While not bound by theory, the above practices are suitable for production of other PTH mid-terminus antibodies using selected mid-terminus PTH sequence peptides as described herein. For example, PTH peptides such as PTH₄₄₋₆₀, PTH₇₋₅₃, PTH₁₂₋₅₃, PTH₁₇₋₅₃, PTH₂₂₋₅₃, PTH₂₇₋₅₃, PTH₃₀₋₃₅, PTH₃₂₋₅₃, PTH₃₂₋₅₃, PTH₃₇₋₅₃, PTH₄₂₋₅₃, PTH₄₇₋₅₃, and other mid-terminal PTH peptides as described above.

In a particularly preferred embodiment, mid-terminus PTH antibodies successfully distinguish between mid-terminus peptides and both initial sequence and C-terminal PTH peptides, such that they specifically bind mid-terminus PTH peptides.

C. Human C-Terminal PTH IRMA Assay

In order to investigate the mechanism of the inhibitory effect and investigate its possible application in clinical procedures, Scantibodies Laboratories, Inc. (Santee, Calif., U.S.A.) has developed a C-terminal PTH IRMA assay in which a goat antibody reacting to the very short PTH C-terminus is used for determination of human PTH molecule(s) with intact C-terminus. Application of this assay provides precise determination of the levels of human PTH molecule(s) with intact C-terminus and facilitates research of bone metabolism regulation.

Scantibodies C-terminal hPTH assay is an immunoradiometric assay (IRMA) utilizing a polyclonal PTH antibody reacting to an epitope within PTH (40-60) (detection antibody), and a polyclonal PTH antibody reacting to an epitope within PTH (75-84) (capture antibody). The epitope recognized by the capture antibody may include the last two amino acids of hPTH, such that only PTH peptides with an intact C-terminus are bound by the antibody. Thus, the use of these antibodies leads to an assay that detects only PTH molecules containing an intact PTH C-terminus.

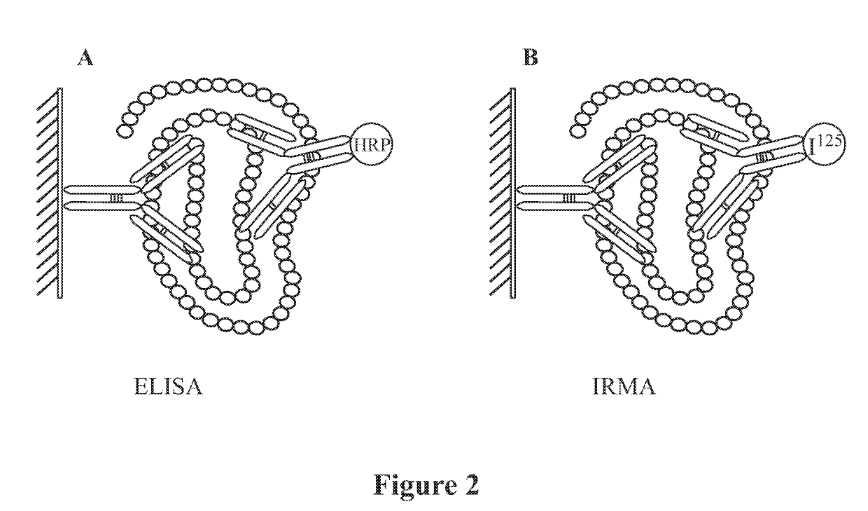

The detection antibody is labeled with I¹²⁵, whereas the capture antibody is immobilized to the beads. The C-terminal PTH molecules in patient samples bind both to the beads and the detection antibody (see FIG. 2B). After incubation, free I¹²⁵ labeled antibodies and bound I¹²⁵ labeled antibody fractions are separated by discarding the supernatant. Simple wash steps reduce the non-specific binding to a minimum for increased precision at the low end of the calibration curve. The concentration of C-terminal PTH is directly proportional to the radioactivity bound to the tubes after separation. The concentration of C-terminal PTH in unknown patient samples and controls is determined by interpolation using a calibration curve.

D. Rat C-Terminal PTH ELISA Assay

The rat is an optimal small animal model to study calcium regulating hormones and bone mineral metabolism. Accordingly, precise determination of rat C-terminal PTH molecule levels would be very helpful in research of bone metabolism regulation.

Scantibodies Laboratories, Inc. (Santee, Calif., U.S.A.) has developed a one-wavelength, two-site Enzyme-Linked Immunosorbent Assay (ELISA) utilizing polyclonal antibodies directed against an epitope within the PTH (40-60) and PTH (75-84) sequences of the rat PTH (parathyroid hormone) peptide. The antibody against PTH (40-60) is used for detection, whereas the antibody against PTH (75-84) is used for capture. The epitope recognized by the capture antibody may include the last two amino acids of hPTH, such that only PTH peptides with an intact C-terminus are bound by the antibody. Thus, the use of these highly specific antibodies ensures that only rat C-terminal PTH molecules with an intact C-terminus are detected.

The rat C-terminal PTH detection antibody is labeled with horseradish peroxidase. The antibody directed against the C-terminal region of rat PTH is immobilized onto the surface of the plastic wells of 96-well ELISA microtiter plates. The rat C-terminal PTH calibrators, controls and unknown rat serum samples are simultaneously incubated with the two antibodies in microtiter plate wells. The C-terminal PTH is captured between the two antibodies and therefore immobilized onto the surface of the plastic wells of the microtiter plates as shown in FIG. 2A.

After incubation, free and bound label antibodies are separated by simple aspiration and washing steps. During the short incubation with the substrate TMB (tetramethylbenzidine) there is a reaction with the peroxidase conjugated to the tracer antibody resulting in a blue color. The addition of a stopping solution converts the color to a stable yellow solution, and the optical absorbance is read immediately at 450 nm. The concentration of rat PTH in the samples is directly proportional to the yellow color intensity. The concentration of PTH in the unknown samples and controls is determined by interpolation from a calibration curve generated using standard cPTH samples.

Expected values of rat serum cPTH were determined using serum samples collected from 33 apparently normal Sprague-Dawley rats (17 female and 16 male, age 6-8 weeks) (see Table 1). These values represent the 95% confidence range.

TABLE 1
Mean serum cPTH values in normal rats and expected cPTH range.

| Serum | No. of | Mean cPTH | Expected |
|---|---|---|---|
| specimens | samples | (pM) | cPTH range (pM) |
| Apparently | 33 | 5.89 | 0-27 |
| normal rats | | | |

E. Changes in Circulating Rat PTH Molecular Forms in Secondary Hyperparathyroidism Related to Vitamin D Deficiency and Renal Failure

The behavior of circulating PTH molecular forms has been studied extensively under a variety of clinical conditions, and their immunoreactivity in second- (Intact or Total hPTH assays) and third- (Whole PTH assay) generation PTH assays is well characterized. In contrast, relatively little is known about the similarity and possible differences between species concerning PTH molecular forms. A study was performed to evaluate the behavior of circulating PTH molecular forms in normal rats (NR) and in rats with secondary hyperparathyroidism related to vitamin D deficiency (DR) and advanced renal failure (RF). Rat PTH assays similar to human assays were developed for this study.

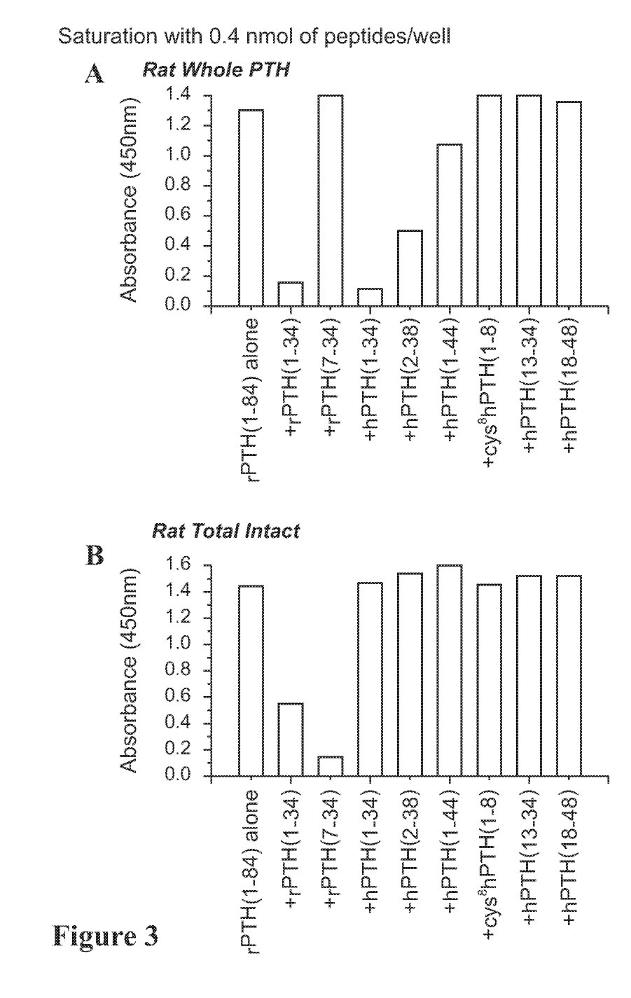

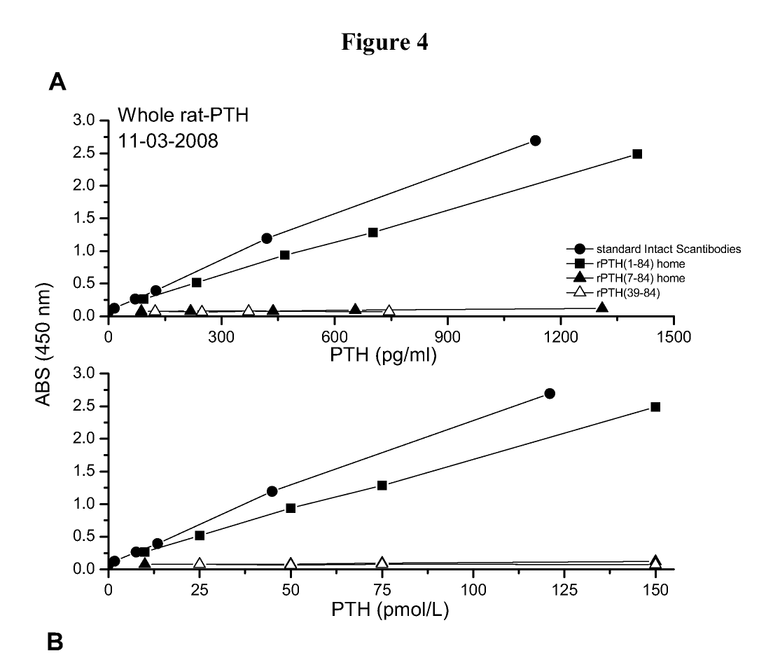

The rat whole PTH (wPTH) assay reacted with rPTH(1-84) but not with rPTH(7-84), demonstrating a (1-6) rPTH epitope (see FIGS. 3A and 4). FIG. 3A shows that rPTH (1-34) and hPTH (1-34) strongly inhibited the wPTH assay, while hPTH (2-38) demonstrated moderate inhibition, indicating that the epitope of the detection antibody used in the whole rPTH assay was part of the rPTH (1-34), and the first amino acid was involved in binding to the detection antibody. The detection antibody in this assay did not react with rPTH (7-34), indicating that the rat whole PTH assay detects only the rPTH with an intact N-terminus. Consistent with these findings, FIG. 4 demonstrates that the wPTH assay did not detect any rPTH (7-84) or rPTH (39-84), and only detected rPTH (1-84).

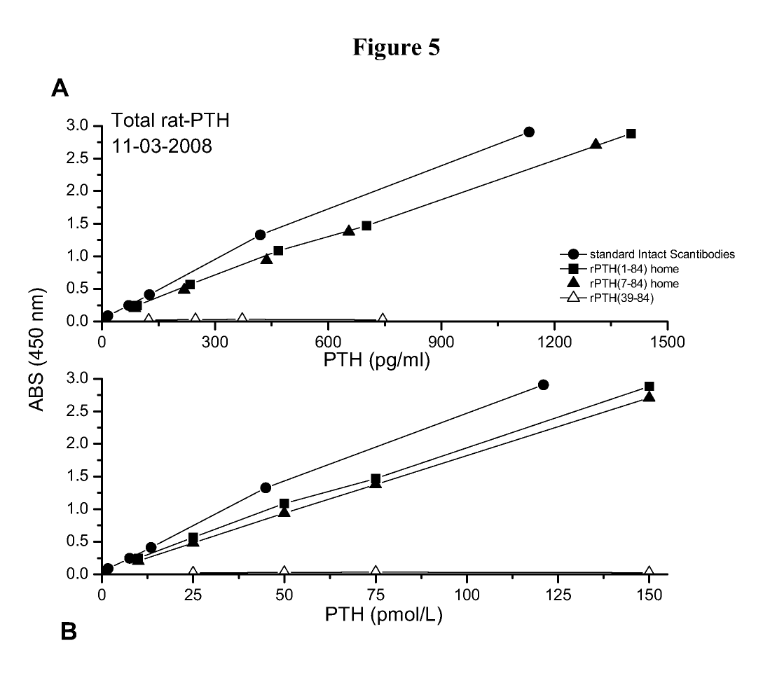

In contrast, the rat total PTH (tPTH) assay reacted equally well with rPTH(1-84) and (7-84) and could be saturated with rPTH (7-34), indicating an epitope in the (7-34) rPTH region (see FIGS. 3B and 5). FIG. 3B demonstrates that the total rPTH assay was not inhibited by hPTH (1-34), and the epitope of the detection antibody used in the total rPTH assay was part of rPTH (7-34) rather than rPTH (1-6). Consistent with these findings, FIG. 5 shows that the tPTH assay detected both rPTH (1-84) and rat PTH (7-84), but did not detect any rPTH (39-84).

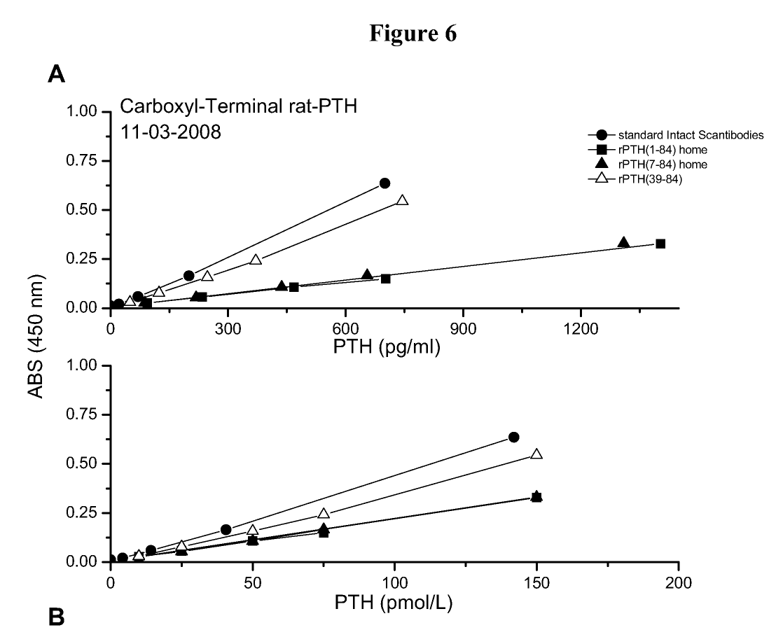

The rat C-terminal PTH assay (cPTH) detected each of rPTH (1-84), rPTH (7-84) and rPTH (39-84) (see FIG. 6). However, the signal from rat PTH (39-84) appeared to be more potent than the signals from rPTH (1-84) and rPTH (7-84), suggesting that the length of a C-terminal fragment affects the sensitivity of the cPTH assay.

The study first revealed that rats with vitamin D deficiency had lower calcium (Ca²⁺) (p<0.05) and 25(OH) vitamin D levels (p<0.001) than normal rats and showed a 3-fold increase in whole rPTH (15±17.6 pmol/L; p<0.001) and total rPTH (20.8±9.9 pmol/L; p<0.001) levels. Similarly, rats with renal failure had a low Ca²⁺ (p<0.01) and a 25 to 30-fold increase in whole rPTH (140±70 pmol/L; p<0.001) and total rPTH (147±72; p<0.001) levels.

Further, whole PTH (wPTH), total PTH (tPTH) and C-terminal PTH (cPTH) were measured in rats of different ages and sexes, and ratios were calculated for tPTH/wPTH, cPTH/wPTH and cPTH/tPTH (see Table 2). The male rats demonstrated elevated PTH ratios across all three age groups. The levels of cPTH fragments strongly exceeded those of wPTH and tPTH in both male and female rats, regardless of age. Since C-terminal PTH fragments have been reported to be associated with certain types of biological activity, correlation of this large amount of cPTH fragments with disease development warrants further investigation. The ratio of cPTH/wPTH appears to provide better assessment for PTH metabolism and possibly correlation with disease development.

TABLE 2
Detection of whole PTH (wPTH), total PTH (tPTH) and C-terminal PTH (cPTH)
in rats.

| | | | | | tPTH/wPTH | cPTH/wPTH | cPTH/tPTH |
|---|---|---|---|---|---|---|---|
| Rat groups | N | wPTH, pM | tPTH, pM | cPTH, pM | ratio | ratio | ratio |
| Male 25 | 25 | 5.33 ± 1.97 | 5.55 ± 1.79 | 10.89 ± 4.70 | 1.08 ± 0.20 | 2.05 ± 0.36 | 1.93 ± 0.41 |
| days | | | | | | | |
| Female 25 | 21 | 5.56 ± 3.89 | 5.06 ± 3.08 | 7.11 ± 5.33 | 0.98 ± 0.20 | 1.33 ± 0.42 | 1.36 ± 0.35 |
| days | | | | | | | |
| Male 50 | 20 | 4.94 ± 2.64 | 7.72 ± 3.12 | 14.17 ± 6.16 | 1.66 ± 0.23 | 2.99 ± 0.71 | 1.81 ± 0.40 |
| days | | | | | | | |
| Female 50 | 22 | 6.82 ± 3.32 | 8.49 ± 3.27 | 10.42 ± 5.59 | 1.32 ± 0.20 | 1.50 ± 0.25 | 1.16 ± 0.25 |
| days | | | | | | | |
| Male 75 | 26 | 4.31 ± 1.87 | 6.77 ± 2.25 | 11.70 ± 6.27 | 1.65 ± 0.25 | 2.65 ± 0.57 | 1.65 ± 0.47 |
| days | | | | | | | |
| Female 75 | 24 | 9.61 ± 3.34 | 10.67 ± 3.09 | 15.92 ± 5.95 | 1.14 ± 0.15 | 1.68 ± 0.29 | 1.47 ± 0.23 |
| days | | | | | | | |

To determine changes in the molecular forms of circulating rPTH in secondary hyperparathyroidism related to vitamin D deficiency and renal failure, pools of serum coming from 25 NR, 13 DR and 5RF animals were subjected to HPLC separation and analyzed using the whole, total and C-terminal rPTH assays. Three regions were identified on HPLC profiles corresponding to fractions 28-37, 38-42 and 43-49. The first corresponded to non-(1-84) rPTH fragments reacting in the rat total PTH assay, the second to N-terminal rPTH reacting slightly better in the rat whole PTH assay, and the third to complete rPTH(1-84).

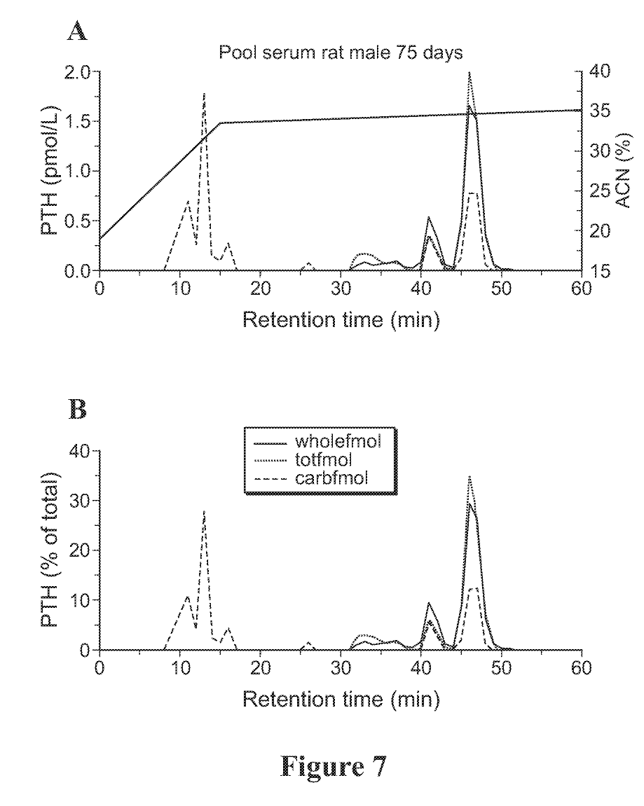

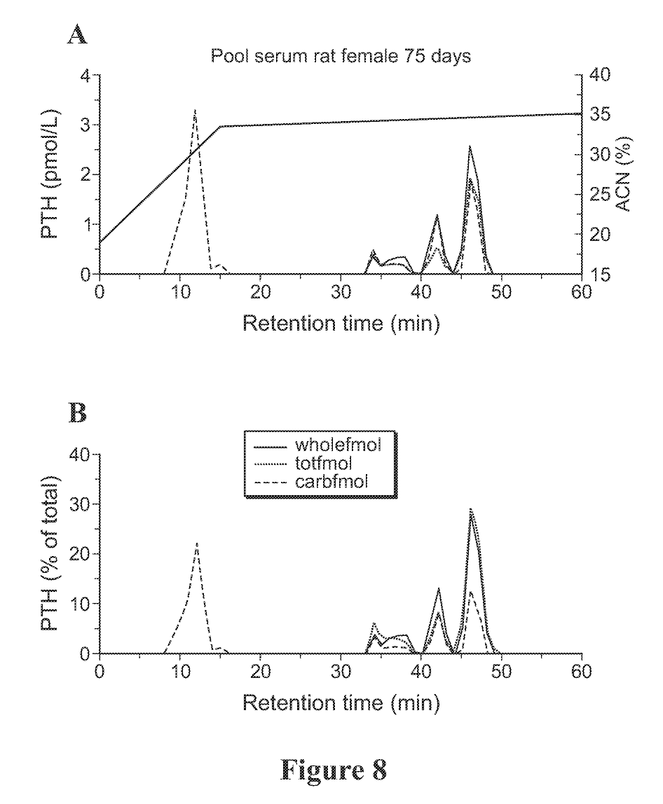

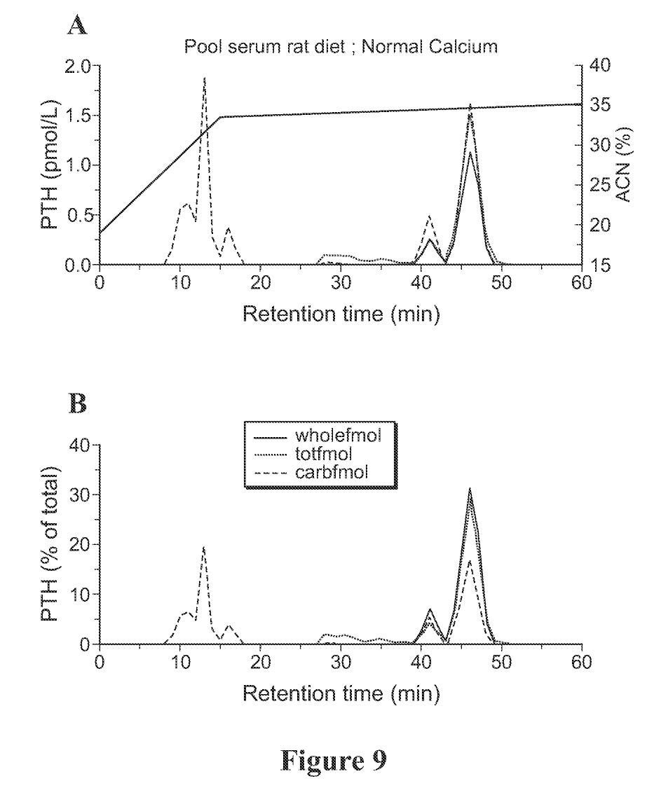

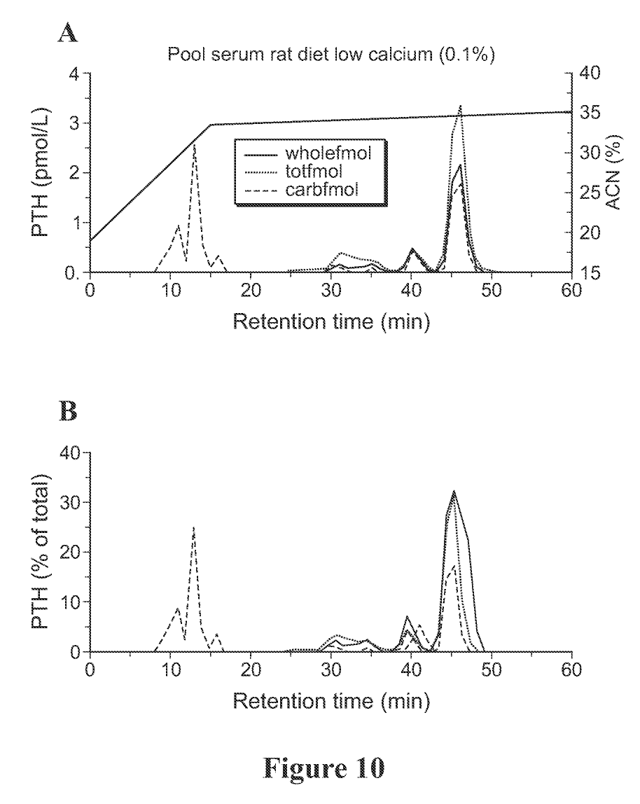

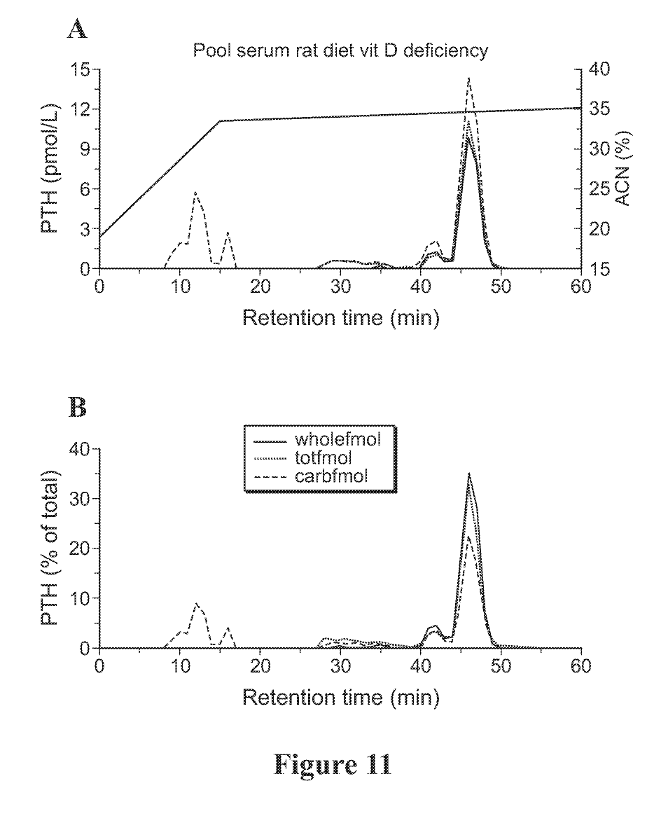

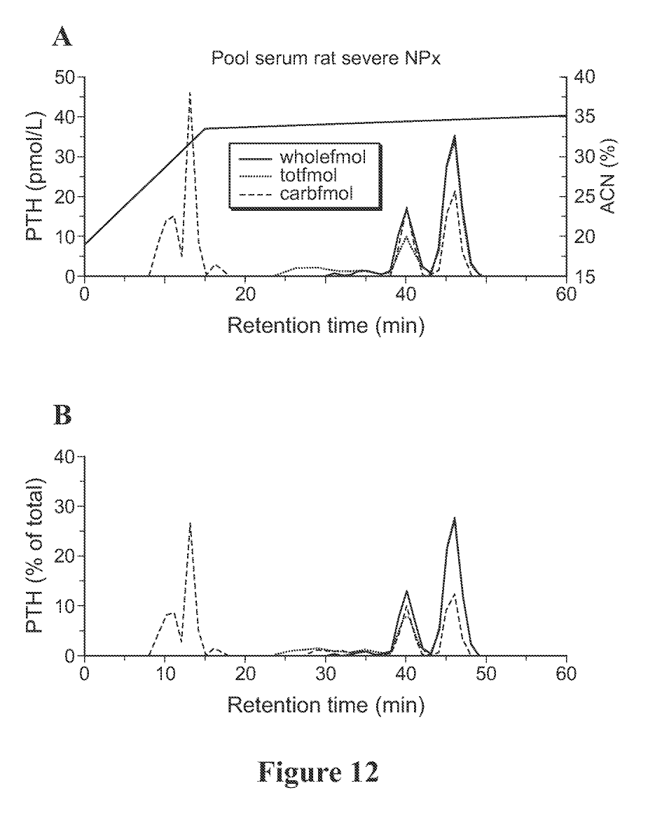

In the whole rPTH assay, rPTH(1-84) accounted for 90.4% and N-PTH for 8.4% of the total in DR animals; 86.4% and 13.6%, respectively, in NR animals; and 67.5% and 29.9%, respectively, in RF animals. The quantity of non-(1-84) PTH was negligible (see FIGS. 7-12 and Table 3).

TABLE 3
rPTH measurements in rat whole PTH assay (wPTH),
as determined by HPLC.

| | Assay total, | non(1-84), | N-PTH, | r(1-84), | | | |
|---|---|---|---|---|---|---|---|
| Rat groups | pM | pM | pM | pM | non(1-84), % | N-PTH, % | r(1-84), % |
| Male 75 days | 5.68 | 0.51 | 1.05 | 4.12 | 9.0 | 18.5 | 72.5 |
| Female 75 days | 9.19 | 1.7 | 2.15 | 5.34 | 18.5 | 23.4 | 58.1 |
| Low calcium | 6.7 | 0.77 | 0.92 | 5.01 | 11.5 | 13.7 | 74.8 |
| Normal calcium | 3.6 | 0.0 | 0.49 | 3.11 | 0.0 | 13.6 | 86.4 |
| Vitamin D | 28.1 | 0.34 | 2.36 | 25.41 | 1.2 | 8.4 | 90.4 |
| deficiency | | | | | | | |
| Renal failure | 127.3 | 3.4 | 38 | 85.9 | 2.7 | 29.9 | 67.5 |

In the total rPTH assay, rPTH (1-84) was 82.5%, N-PTH 6.4% and non-(1-84) PTH 11.1% in DR animals; 78.9%, 9.6% and 11.5%, respectively, in NR animals; and 68.3%, 19.5% and 12.2%, respectively, in RF animals (see FIGS. 7-12 and Table 4).

TABLE 4
rPTH measurements in rat total PTH assay (tPTH), as determined by HPLC.

| | Assay total, | non(1-84), | N-PTH, | r(1-84), | | | |
|---|---|---|---|---|---|---|---|
| Rat groups | pM | pM | pM | pM | non(1-84), % | N-PTH, % | r(1-84), % |
| Male 75 days | 5.81 | 0.76 | 0.65 | 4.4 | 13.1 | 11.2 | 75.7 |
| Female 75 days | 6.8 | 1.31 | 1.18 | 4.31 | 19.3 | 17.4 | 63.4 |
| Low calcium | 11.1 | 2.09 | 1.13 | 7.88 | 18.8 | 10.2 | 71.0 |
| Normal calcium | 5.4 | 0.62 | 0.52 | 4.26 | 11.5 | 9.6 | 78.9 |
| Vitamin D | 34 | 3.77 | 2.19 | 28.04 | 11.1 | 6.4 | 82.5 |
| deficiency | | | | | | | |
| Renal failure | 122.1 | 14.9 | 23.8 | 83.4 | 12.2 | 19.5 | 68.3 |

In the C-terminal PTH assay, rPTH(1-84) was 58.1%, N-PTH 7.1%, non-(1-84) PTH 6.1% and cPTH was 28.6% in DR animals; 41.1%, 10.0%, 0.6% and 48.3%, respectively, in NR animals; and 25.3%, 17.6%, 0% and 57.1%, respectively, in RF animals (see FIGS. 7-12 and Table 5).

TABLE 5
rPTH measurements in rat C-terminal PTH assay (cPTH), as determined by
HPLC.

| | | cPTH | | | | | | | |
|---|---|---|---|---|---|---|---|---|---|
| Rat | Assay | frag., | non(1-84), | N-PTH, | r(1-84), | cPTH | | | |
| groups | total, pM | pM | pM | pM | pM | frag., % | non(1-84), % | N-PTH, % | r(1-84), % |
| Male 75 | 6.4 | 4.03 | 0.09 | 0.53 | 1.75 | 63.0 | 1.4 | 8.3 | 27.3 |
| days | | | | | | | | | |
| Female 75 | 14.9 | 8.24 | 1.38 | 1.89 | 3.39 | 55.3 | 9.3 | 12.7 | 22.8 |
| days | | | | | | | | | |
| Low | 10.2 | 5.45 | 0.35 | 0.68 | 3.72 | 53.4 | 3.4 | 6.7 | 36.5 |
| calcium | | | | | | | | | |
| Normal | 9.4 | 4.54 | 0.06 | 0.94 | 3.86 | 48.3 | 0.6 | 10.0 | 41.1 |
| calcium | | | | | | | | | |
| Vitamin D | 63.2 | 18.1 | 3.84 | 4.46 | 36.8 | 28.6 | 6.1 | 7.1 | 58.2 |
| deficiency | | | | | | | | | |
| Renal | 171 | 97.6 | 0.00 | 30.1 | 43.3 | 57.1 | 0.0 | 17.6 | 25.3 |
| failure | | | | | | | | | |

The results indicate that the adjustment of circulating PTH molecular forms in rats is mainly accomplished through rPTH(1-84) and N-PTH and less through non-(1-84) PTH fragments, with a decreased production of N-PTH in favor of rPTH(1-84) in vitamin D deficiency and with the accumulation of N-PTH in renal failure. Moreover, the results from the C-terminal PTH assay indicate that the rats with vitamin D deficiency and the rats with renal failure had higher serum cPTH concentrations. Notably, C-terminal PTH fragments accounted for about 50% or more in all the animal groups tested, except for the rats maintained on a vitamin D deficient diet.

The ordinarily skilled artisan can appreciate that the present invention can incorporate any number of the preferred features described above.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. An isolated antibody that specifically binds to a C-terminal sequence of whole parathyroid hormone (PTH); preferably, the isolated antibody is capable of detecting said whole PTH, a non-whole PTH fragment and/or a C-terminal PTH fragment at a physiological level in a mammalian sample, and/or specifically binds to a whole PTH, a non-whole PTH fragment and/or a C-terminal PTH fragment having an intact C-terminus.

2. The isolated antibody of claim 1, which is a monoclonal or polyclonal antibody or antibody fragment.

3. The isolated antibody of claim 1, which specifically binds to an epitope comprised in PTH₈₁₋₈₄, PTH₈₀₋₈₄, PTH₇₉₋₈₄, PTH₇₈₋₈₄, PTH₇₇₋₈₄, PTH₇₆₋₈₄ or PTH₇₅₋₈₄.

4. The isolated antibody of claim 1, which specifically binds to the parathyroid hormone peptide human PTH₇₅₋₈₄, rat PTH₇₅₋₈₄, mouse PTH₇₅₋₈₄, bovine PTH₇₅₋₈₄, canine PTH₇₅₋₈₄, porcine PTH₇₅₋₈₄ or horse PTH₇₅₋₈₆, wherein at least four amino acids in said peptide sequence are part of a reactive portion with the antibody.

5. A method for measuring the level of a whole PTH, a non-whole PTH fragment and/or a C-terminal PTH fragment having an intact C-terminus in a mammalian sample, which method comprises:
a) obtaining a sample from a mammal to be tested;
b) contacting said sample with an isolated antibody that specifically binds to a C-terminal sequence of whole PTH, preferably, the isolated antibody being capable of detecting said whole PTH, non-whole PTH fragment and/or C-terminal PTH fragment at a physiological level in said mammalian sample, and/or specifically binding to said whole PTH, non-whole PTH fragment and/or C-terminal PTH fragment having an intact C-terminus; and
c) assessing a complex formed between said whole parathyroid hormone, non-whole PTH fragment and/or C-terminal PTH fragment, if present in said sample, and said antibody, to measure the level of said whole PTH, non-whole PTH fragment and/or C-terminal PTH fragment in said mammalian sample.

6. The method of claim 5, wherein the sample is selected from the group consisting of a serum, a plasma and a blood sample.

7. The method of claim 5, wherein the sample is a clinical sample.

8. The method of claim 5 which is used for clinical management of renal disease subjects, subjects afflicted with osteoporosis or diagnosing primary hyperparathyroidism.

9. The method of claim 5, wherein the mammal is a human.

10. The method of claim 9, wherein the sample is a human clinical sample.

11. The method of claim 5, wherein the antibody is a monoclonal or polyclonal antibody or antibody fragment.

12. The method of claim 5, wherein the antibody specifically binds to an epitope comprised in PTH₈₁₋₈₄, PTH₈₀₋₈₄, PTH₇₉₋₈₄, PTH₇₈₋₈₄, PTH₇₇₋₈₄, PTH₇₆₋₈₄, PTH₇₅₋₈₄, PTH₇₄₋₈₄, PTH₇₃₋₈₄, PTH₇₂₋₈₄, PTH₇₁₋₈₄ or PTH₇₀₋₈₄.

13. The method of claim 5, wherein the antibody specifically binds to the parathyroid hormone peptide human PTH₇₅₋₈₄, rat PTH₇₅₋₈₄, mouse PTH₇₅₋₈₄, bovine PTH₇₅₋₈₄, canine PTH₇₅₋₈₄, porcine PTH₇₅₋₈₄ or horse PTH₇₅₋₈₄, wherein at least four amino acids in said peptide sequence are part of a reactive portion with the antibody.

14. The method of claim 5, wherein the complex is assessed by a sandwich or competitive assay format.

15. The method of claim 14, wherein the antibody that specifically binds to a C-terminal sequence of whole PTH is used as a first antibody and an antibody that is capable of binding to a portion of the whole PTH, non-whole PTH fragment and/or C-terminal PTH fragment other than the C-terminal sequence which binds to the first antibody is used as a second antibody in a sandwich assay format.

16. The method of claim 15, wherein either the first antibody or the second antibody is attached to a surface and functions as a capture antibody.

17. The method of claim 16, wherein the capture antibody is attached to the surface directly or indirectly.

18. The method of claim 16, wherein the capture antibody is attached to the surface via a biotin-avidin (or streptavidin) linking pair.

19. The method of claim 5, wherein the complex is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, u-capture assay, inhibition assay and avidity assay.

20. The method of claim 5, wherein the complex is assessed in a homogeneous or a heterogeneous assay format.

21. The method of claim 5, wherein the physiological level of whole parathyroid hormone is less than 4 pmol/L.

22. The method of claim 5, wherein the physiological level of whole parathyroid hormone is from about 0.2 pmol/L to about 4 pmol/L.

23. The method of claim 15, wherein the second antibody specifically binds to an epitope comprised within PTH₄₀₋₆₀, PTH₃₅₋₄₀, PTH₅₃₋₇₃, or PTH₃₅₋₇₄ sequence.

24. The method of claim 23, wherein the second antibody specifically binds to an epitope comprised within PTH₄₀₋₆₀ sequence.

25. The method of claim 23, wherein at least one of the first or second antibody is labeled.

26. The method of claim 5, which further comprises measuring a whole PTH level and/or a total PTH level.

27. The method of claim 26, which further comprises comparing at least two parameters selected from the group consisting of the whole PTH level, the total PTH level, and the C-terminal PTH level.

28. The method of claim 27, wherein the results of said comparison are used to determine whether the mammal suffers from a bone turnover related disorder, or to monitor bone disease or disorder related treatment.

29. The method of claim 28, which is used in the diagnosis or monitoring of treatment for adynamic bone disease (ADN) or severe hyperparathyroidism.

30. The method of claim 27, wherein the results of said comparison are used to determine whether the mammal suffers from a chronic kidney failure, or to monitor chronic kidney failure related treatment.

31. The method of claim 27, wherein the comparison is in the form of a ratio or proportion between the C-terminal PTH level and the whole PTH level.

32. The method of claim 27, wherein the comparison is in the form of a ratio or proportion between the C-terminal PTH level and the total PTH level.

33. The method of claim 5, which is used for:
a) differentiating between a person having substantially normal parathyroid function and having hyperparathyroidism;
b) monitoring parathyroid related bone disease and treatment;
c) monitoring effects of therapeutic treatment for hyperparathyroidism; or
d) diagnosing parathyroid related bone disease.

34. The method of claim 5, which is used for:
a) differentiating between a person having substantially normal renal function and having chronic renal failure;
b) monitoring chronic renal failure;
c) monitoring effects of therapeutic treatment for chronic renal failure; or
d) diagnosing chronic renal failure.

35. A kit for measuring the level of a whole PTH, a non-whole PTH fragment and/or a C-terminal PTH fragment having an intact C-terminus in a mammalian sample, which kit comprises, in a container, an isolated antibody that specifically binds to a C-terminal sequence of whole parathyroid hormone (PTH); preferably, the isolated antibody is capable of detecting said whole PTH, a non-whole PTH fragment and/or a C-terminal PTH fragment at a physiological level in a mammalian sample, and/or specifically binds to a whole PTH, a non-whole PTH fragment and/or a C-terminal PTH fragment having an intact C-terminus.

36. An isolated parathyroid hormone (PTH) peptide, which is selected from the group consisting of PTH₈₁₋₈₄, PTH₈₀₋₈₄, PTH₇₉₋₈₄, PTH₇₈₋₈₄, PTH₇₇₋₈₄, PTH₇₆₋₈₄, PTH₇₅₋₈₄, PTH₇₄₋₈₄, PTH₇₃₋₈₄, PTH₇₂₋₈₄, PTH₇₁₋₈₄ or PTH₇₀₋₈₄.

37. The isolated PTH peptide of claim 36, wherein the peptide is PTH₇₅₋₈₄.

38. The isolated PTH peptide of claim 36, which is conjugated to a carrier to enhance the PTH peptide's immunogenicity.

39. The isolated PTH peptide of claim 38, wherein the carrier is a carrier protein.

40. The isolated PTH peptide of claim 39, wherein the PTH peptide and the carrier protein are parts of a fusion protein.

41. An immunogen, which immunogen comprises:
a) a PTH peptide of claim 36; and
b) an immune response potentiator.

42. The immunogen of claim 41, wherein the immune response potentiator is selected from the group consisting of Bacille Calmette-Guerin (BCG), Corynebacterium Parvum, Brucella abortus extract, glucan, levamisole, tilorone, an enzyme and a non-virulent virus.

43. A multiple antigenic peptide (MAP), which MAP comprises a branched oligolysine core conjugated with a plurality of the PTH peptide of claim 36.

44. The MAP of claim 43, wherein the branched oligolysine core comprises 3, 7 or 15 lysine residues.

45. The MAP of claim 43, wherein the plurality of the PTH peptide is conjugated to the branched oligolysine core via a spacer.

46. The MAP of claim 45, wherein the spacer is an amino acid residue.

47. The MAP of claim 45, which comprises 4, 8 or 16 copies of the PTH peptide.

48. The MAP of claim 43, wherein the plurality of the PTH peptide comprises same or different PTH peptides.

49. A method for producing an antibody to a parathyroid hormone (PTH) or a PTH peptide, which method comprises:
a) introducing an isolated PTH peptide of claim 36 to a mammal in an amount sufficient to produce an antibody to said PTH peptide; and
b) recovering said antibody from said mammal.

50. An antibody to a PTH or a PTH peptide produced by the method of claim 49.

51. A kit for producing an antibody to a parathyroid hormone (PTH) or a PTH peptide, which kit comprises:
a) an isolated PTH peptide of claim 36;
b) means for introducing said isolated PTH peptide to a mammal in an amount sufficient to produce an antibody to said PTH peptide; and
c) means for recovering said antibody from said mammal.

52. A method for producing an antibody to a parathyroid hormone (PTH) or a PTH peptide, which method comprises:
a) introducing a MAP of claim 43 to a mammal in an amount sufficient to produce an antibody to a PTH peptide comprised in said MAP; and
b) recovering said antibody from said mammal.

53. An antibody to a PTH or a PTH peptide produced by the method of claim 52.

54. A kit for producing an antibody to a parathyroid hormone (PTH) or a PTH peptide, which kit comprises:
a) a MAP of claim 43;
b) means for introducing said MAP to a mammal in an amount sufficient to produce an antibody to a PTH peptide comprised in said MAP; and
c) means for recovering said antibody from said mammal.

55. A method for producing an antibody to a parathyroid hormone (PTH) or a PTH peptide, which method comprises:
a) introducing a PTH protein or peptide from between PTH₁₋₈₄ and PTH₃₅₋₈₄ to a mammal in an amount sufficient to produce an antibody to said PTH protein or peptide;
b) recovering said antibody from said mammal; and
c) affinity purifying a PTH antibody that specifically binds to an epitope comprised in a PTH peptide of claim 36 using said PTH peptide.

56. An antibody to a PTH or a PTH peptide produced by the method of claim 55.

57. A kit for producing an antibody to a parathyroid hormone (PTH) or a PTH peptide, which kit comprises:
a) a PTH protein or peptide from between PTH₁₋₈₄ and PTH₃₅₋₈₄;
b) means for introducing said PTH protein or peptide from between PTH₁₋₈₄ and PTH₃₅₋₈₄ to a mammal in an amount sufficient to produce an antibody to said PTH protein or peptide;
c) means for recovering said antibody from said mammal; and
d) a PTH peptide of claim 36.

58. The method of claim 5, wherein the physiological level of whole parathyroid hormone is from about 7 pg/ml to about 39 pg/ml.
